(19) United States
(12) Patent Application Publication
(10) Pub. No.: US 2003/0049831 A1
(43) Pub. Date: Mar. 13, 2003

(54) Novel factor-H related protein 5 and antibodies thereto

(76) Inventor: Brendan F. Murphy, Victoria (AU)

Correspondence Address: Charles R. Mattenson, Corp. Counsel, Renal Div., Baxter Healthcare Corp., One Baxter Parkway, DF3-3E, Deerfield, IL, 60015-4633, US

(21) Appl. No.: 09/805,337

(22) Filed: Mar. 13, 2001

Related U.S. Application Data
(60) Provisional application No. 60/188,870, filed on Mar. 13, 2000.

(51) Int. Cl. C07H 21/02; C07H 21/04; C12N 1/20; C07K 1/00; C07K 14/00; C07K 17/00

(52) U.S. Cl. 435/252.3; 536/23.1; 530/350

(57) ABSTRACT

A novel human factor H-related protein (FHR-5) is disclosed. Included within the scope of the invention are the amino acid sequence of FHR-5, nucleic acid sequences encoding the amino acid sequence of FHR-5 and substantially identical amino acid and nucleotide sequences. Also within the scope of the invention are vectors comprising the nucleotide sequences of the invention, transgenic host cells containing the nucleotide sequences of the invention and antibodies that react with the FHR-5 protein.

### CROSS-REFERENCE TO RELATED APPLICATIONS

This application claims the benefit of U.S. Provisional Patent Application Serial No. 60/188,870, filed Mar. 13, 2000, which is herein incorporated by reference in its entirety for all purposes.

### BACKGROUND

The present invention is directed towards a novel human protein, Factor-H related Protein 5 (FHR-5), proteins substantially identical to FHR-5, and antibodies directed to these proteins.

The action of complement is the principal means by which antibodies defend vertebrates against most bacterial infections. Complement consists of a system of serum proteins that can be activated by antibody-antigen complexes or microorganisms to undergo a cascade of proteolytic reactions whose end result is the assembly of membrane attack complexes. These complexes form holes in a microorganism and thereby destroy it. At the same time, proteolytic fragments released during the activation process promote the defense response by dilating blood vessels and attracting phagocytic cells to sites of infection. Complement also enhances the ability of phagocytic cells to bind, ingest, and destroy the microorganisms being attacked.

Complement consists of about 20 interacting proteins, of which the reacting components are designated C1-C9, factor B, and factor D, and wherein the rest comprise a variety of regulatory proteins, including the factor H proteins. The complement components are all soluble proteins. They are made mainly by the liver and circulate in the blood and extracellular fluid. Most are inactive unless they are triggered directly by an invading microorganism or indirectly by an immune response. The ultimate consequence of complement activation is the assembly of the late complement components (C5, C6, C7, C8, and C9) into a large protein complex (the membrane attack complex) that mediates microbial cell lysis.

Because its main finction is to attack the membrane of microbial cells, the activation of complement is focused on the microbial cell membrane, where it is triggered either by antibody bound to the microorganism or by microbial envelope polysaccharides, both of which activate the early complement components. There are two sets of early components belonging to two distinct pathways of complement activation: C1, C2, and C4 belong to the classical pathway, which is triggered by antibody binding; factor B and factor D belong to the alternative pathway, which is triggered by microbial polysaccharides. The early components of both pathways ultimately act on C3, the most important complement component. The early components and C3 are proenzymes that are activated sequentially by limited proteolytic cleavage: as each proenzyme in the sequence is cleaved, it is activated to generate a serine protease, which cleaves the next proenzyme in the sequence, and so on. Many of these cleavages liberate a small peptide fragment and expose a membrane-binding site on the larger fragment. The larger fragment binds tightly to the target cell membrane by its newly exposed membrane-binding site and helps to carry out the next reaction in the sequence. In this way, complement activation is confined largely to the cell surface where it began.

The activation of C3 by cleavage is the central reaction in the complement-activation sequence, and it is here that the classical and alternative pathways converge. In both pathways, C3 is cleaved by an enzyme complex called a C3 convertase. A different C3 convertase is produced by each pathway, formed by the spontaneous assembly of two of the complement components activated earlier in the cascade. Both types of C3 convertase cleave C3 into two fragments. The larger of these (C3b) binds covalently to the target-cell membrane and binds C5. Once bound, the C5 protein is cleaved by the C3 convertase (now acting as a C5 convertase) to initiate the spontaneous assembly of the late components (C5 through C9) that creates the membrane attack complex. Since each activated enzyme cleaves many molecules of the next proenzyme in the chain, the activation of the early components consists of an amplifying proteolytic cascade, each molecule activated at the beginning of the sequence leads to the production of many membrane attack complexes.

Assembly of the late components begins when C5, already loosely bound to C3b on the target cell membrane, is split by the C3 convertase of either the classical or alternative pathway to give C5a and C5b. As just described, the C5a is released and promotes an inflammatory response. The C5b remains bound to the C3b and has the transient capacity to bind C6 to form C56 and then C7 to form C567. The C567 complex then binds firmly via C7 to the membrane. This complex adds one molecule of C8 to form C5678, which then binds 8 to 18 molecules of C9, which partially unfold and polymerize into a trans-membrane channel.

The self-amplifying destructive properties of the complement cascade make it essential that key activated components be rapidly inactivated after they are generated to ensure that the attack does not spread to nearby host cells. Deactivation is achieved in at least two ways. First, specific inhibitor proteins in the blood terminate the cascade by either binding or cleaving certain components once they have been activated by proteolytic cleavage. Inhibitor proteins, for example, bind to the activated components of the C1 complex and block their further action, while other inhibitor proteins cleave C3b and thereby inactivate it. Without these inhibitors, all of the serum C3 might be depleted by the positive feedback loop created by the alternative pathway.

Factor H (FH) (Ripoche et al., Biochem. J. 249:593-602:1988) is a fluid phase RCA (regulators of complement activation) protein encoded by a gene approximately 7 Mb from the main RCA gene cluster on chromosome 1q32 (Hourcade et al., Adv. Immunol., 45:381-416, 1989). It is composed of 20 short consensus repeats (SCR) domains and it functions to prevent amplification of the alternative complement activation pathway by accelerating the decay of C3 and C5 convertases and by acting as a cofactor for factor I-mediated cleavage of surface bound C3b. SCRs 1-10 and 16-20 of FH are believed to contain the binding site(s) for C3b, and decay-accelerating and cofactor activity has been localized to SCR 1-5(Alsenz et al., Biochem. J. 224:389-398, 1984; Alsenz et al., Biochem. J. 232:841-850, 1985; Gordon et al., J. Immunol. 155:348-356, 1995; Kuhn et al., J. Immunol 155:5663-5670, 1995; Kuhn and Zipfel. Euro. J. Immunol., 26:2383-2387, 1996; Soames and Sim, Biochem. Soc. Trans. 23:53S, 1995; Sharma and Pangburn, Proc. Natl. Acad. Sci. USA 93:10996-11001, 1996). The factor H family also includes factor H-like protein 1 (FHL-1), a shorter (SCRs 1-7) version of FH generated by alternative splicing (Schwaeble et al., Euro. J. Immunol., 17:1485-1489, 1987), and the four factor H-related proteins FHR-1 to FHR-4, which contain 4 or 5 SCRs (Zipfel and Skerka, Immunol. Today, 15:121-126, 1994; Skerka et al, J. Biol. Chem., 272:5627-5634, 1997). Of these alternative forms, only FHL-1 has complement regulatory activity, although all contain SCRs implicated in C3b binding.

In studies designed to identify novel components of glomerular immune deposits, a series of monoclonal antibodies was produced after immunization of mice with glomerular basement membrane preparations from human kidneys with glomerulonephritis. Although most of the resulting antibodies were directed against non-structural components, several were reactive with components of the terminal complement complex (Murphy and d'Apice, Pathology, 20:130-136, 1988). These included two antibodies that first identified human clusterin, a component of the SC5b-9 complement complex. The pattern of immunohistological reactivity of another antibody against glomerular immune deposits, K2.254, appeared similar to that of antibodies against components of the terminal complement complex in normal and glomerulonephrotic human tissue. However, this antibody does not react with any known complement component or purified terminal complement C5b-9 complexes, and it does not react with normal human serum. Thus, this antibody was originally thought to bind to a newly exposed epitope on the activated complement C5b-9 complex, putatively on the activated C9 protein, or to an epitope on a factor specific for glomerulonephrosis.

Applicants have discovered that the antibody binds to a heretofore unknown complement associated protein. The deduced amino acid sequence of the protein, obtained by partial peptide sequencing and cDNA cloning and sequencing, indicates that it is a member of the factor H-related family of proteins. In keeping with current nomenclature in this area, the new protein has been designated factor H-related protein 5 (FHR-5).

### SUMMARY

The present invention is directed towards a novel human factor H-related protein 5 (FHR-5), substantially identical proteins, and antibodies to these proteins.

In one aspect, the present invention is drawn to a substantially purified protein whose amino acid sequence is identical to FHR-5, SEQ ID NO. 2. In further embodiments, the present invention is also directed towards proteins which are substantially identical to FHR-5. Thus, in one embodiment such proteins have at least 90% sequence identity to FHR-5 (or about 495 matching amino acids), as measured by the number of identical amino acids in the sequences when the sequence of the protein is aligned with SEQ ID NO. 2, and gaps are introduced in order to produce the greatest number of amino acid matches. In another embodiment, such proteins have at least 95% sequence identity to FHR-5 (or about 523 matching amino acids). In still another embodiment, such proteins have at least 98% sequence identity to FHR-5 (or about 539 matching amino acids), and in yet another embodiment such proteins have at least 99% sequence identity to FHR-5 (or about 545 matching amino acids). In a further embodiment, such proteins have at least 99.5% sequence identity to FHR-5 (or about 548 matching amino acids).

In another aspect, the present invention is drawn to substantially isolated nucleic acid polymers encoding the above proteins. One embodiment of these nucleic acids is the native cDNA sequence of FHR-5, SEQ ID NO. 1, its complement and sequences which hybridize to SEQ ID NO. 1 or its complement under high stringency conditions. Other embodiments of these nucleic acid sequences are the synonymous nucleic acid sequences which also encode SEQ ID NO. 2. These sequences are described by the generic sequence in SEQ ID NO. 3. Also contemplated as embodiments of these sequences are those nucleic acid polymers which encode proteins which are substantially identical to FHR-5. These sequences are those which encode proteins with at least 90% identity to FHR-5, as well as those which encode proteins with at least 95% identity, 98% identity, 99% identity, or 99.5% identity to FHR-5, as defined above. Such sequences may be readily devised and produced by persons of ordinary skill in the art by resorting to the genetic code and utilizing various site directed mutagenesis techniques to derivatize the nucleic acid sequence of SEQ ID NO. 1.

In another aspect, the present invention is also drawn to antibodies to FHR-5 or substantially identical proteins, wherein the antibody is not K2.254. Preferred embodiments of this aspect are non-K2.254 monoclonal antibodies to epitopes of FHR-5 which are selectively displayed when FHR-5 is associated with activated complement. Other preferred embodiments of this aspect are non-K2.254 antibodies to FHR-5 which, when bound to FHR-5, inhibit functions of FHR-5 necessary for the protein to associate with activated complement. An additional embodiment of this aspect is a humanized monoclonal antibody to FHR-5 or a substantially identical protein. Further aspects include recombinant and chimeric antibodies to FHR-5 or a substantially identical protein and immunologically active antibody fragments (e.g. Fab fragments) to FHR-5 or a substantially identical protein.

In another aspect, the present invention is also drawn to methods of utilizing the antibodies of the present invention. The non-K2.254 monoclonal antibodies to an epitope of FHR-5 which is selectively displayed when FHR-5 is associated with activated complement can be used as immunohistochemical diagnostic reagents to detect immunological deposits in biopsied tissues. In addition, non-K2.254 antibodies to FHR-5 which, when bound to FHR-5, inhibit functions of FHR-5 necessary for the protein to associate with activated complement may be used to prevent the association of FHR-5 with activated complement.

In yet another aspect, the present invention is drawn to an isolated polynucleotide selected from the group consisting of: (a) the polynucleotide of SEQ ID NO. 1 and the complement thereof; and (b) a polynucleotide that hybridizes to the polynucleotide of (a) under wash conditions of 0.1×SSPE, 0.1% SDS at 42° C., and which encodes a protein that is recognized by antibody K2.254. A further embodiment provides a oligonucleotide of at least 20 nucleic acids that hybridizes to any of the polynucleotides of the present invention under wash conditions of 0.1×SSPE, 0.1% SDS at 42° C.

### BRIEF DESCRIPTION OF THE FIGURES

These and other features, aspects, and advantages of the present invention will become better understood with regard to the following description, appended claims and accompanying figures where:

FIG. 1 shows immunofluorescence of antibody-sensitized, normal human serum-treated K562 cells stained with K562 Mab. Cells were incubated progressively with polyclonal anti-K562 and normal human serum, and K2.254 and FITC-labelled secondary antibody was used to detect K2.254 antigen binding. Magnification, X400.

FIG. 2 shows Western blots of membranes from complement-lysed HE and GPE. The blot was probed with the K2.254 Mab and ¹²⁵I labelled second antibody. A band at ˜67 kD (arrow) was detected in human complement lysed human erythrocytes (lanes 1 & 2) and guinea pig erythrocytes (lane 3) but not in osmotically lysed human erythrocytes (lane 4).

FIG. 3 shows Western blots of normal (N) and zymosan-activated (Z) human serum. Blots were probed with the anti-native C9 Mab K2.322, the K2.254 Mab or secondary ¹²⁵I antibody alone as a negative control. Migration of Mr marker proteins are indicated on the left. K2.322 (FIG. 3A) detects monomeric C9 (arrow) in both Z and N human serum and dimeric C9 (arrowhead) in the Z human serum, showing that complement activation has occurred. K2.254 (FIG. 3B) shows no specific reactivity with either serum preparation

FIG. 4 shows Western blots of affinity-purified protein extracted from complement-lysed GPE. The affinity-purified preparation contained contaminating C9 (lane 2) and HSA (lane 3) in addition to K2.254 antigen (lane 1). Mr markers are indicated on the left.

FIG. 5 shows schematic representation of FHR-5 cDNA. The ORF is boxed, with the signal peptide encoding region unhatched. cDNA clones generated by RT-PCR are indicated.

FIG. 6 shows homology within the human FH family. (Adapted from Zipfel & Skerka, Immunol Today, 15:121-126, 1994). SCRs of the individual proteins are numbered consecutively. Related SCRs are shown by vertical alignment and gaps are indicated by a dotted line. Distinct SCRs and distinct sequences within SCRs are shown in black and their vertical alignment also indicates homology. Proposed functional domains are represented by horizontal bars: (A), decay accelerating and Co-factor activity; (B), C3b binding; (C), Heparin binding. An RGD sequence has been located to SCR 4 of FH and FHL-1.

FIG. 7 shows Northern blot analysis with human liver RNA. Total cellular RNA and mRNA was isolated from human liver tissue. Ten mg of total RNA and 2mg of mRNA was separated on a denaturing agarose gel and transferred onto nitrocellulose membranes. A ³²P-labelled cDNA probe specific for FHR-5 was used and hybridized to a mRNA species of estimated size 3.0 kilobases. The same membrane was stripped and probed with a cDNA probe specific for FH. This probe detected the characteristic 4.4 kilobase mRNA and a higher 5.0 kb species but did not cross-hybridize with the 3.0 kb species.

FIG. 8 shows Western blot analysis of the culture medium from Sf9 insect cells expressing recombinant FHR-5 (rFHR-5). Proteins were separated under non-reducing and reducing conditions on a 7.5% SDS-PAGE gel and transferred to nitrocellulose membrane for Western blot. To detect expression of the rFHR-5 protein, blots were probed with the K2.254 (lanes 1, non-reduced and 2, reduced) or anti-Tetra-his antibodies (lanes 3, non-reduced and 4, reduced). The anti-C9 monoclonal, K2.322, was used as a negative control and is indicated in lanes 5, non-reduced and 6, reduced. Mr markers are indicated on the left.

FIG. 9 shows the SCR structure of FHR-5. The sequences were aligned based on their conserved amino acids according to the SCR structure. The characteristic cysteine residues are boxed and the conserved sequences are aligned.

FIG. 10 shows the alignment of the SCRs of FHR-5 with other family members. Identical amino acids are shown by dots, individual lines represent the SCRs of FHR-5 and those of the FH family that show greater than 40% homology.

FIG. 11 shows the binding of recombinant FHR-5 in vitro. Binding of C3b, C5b-6 and HSA to recombinant FHR-5 (panel A) or Factor H (panel B) was determined by ELISA.

### DEFINITIONS

“Substantially pure” or “substantially purified” typically means that the substance is free from other contaminating proteins, nucleic acids, and other biologicals derived from the original source organism. Purity may be assayed by standard methods, and will ordinarily be at least about 40% pure, more ordinarily at least about 50% pure, generally at least about 60% pure, more generally at least about 70% pure, often at least about 75% pure, more often at least about 80% pure, typically at least about 85% pure, more typically at least about 90% pure, preferably at least about 95% pure, more preferably at least about 98% pure, and in even more preferred embodiments, at least 99% pure. The analysis may be weight or molar percentages, evaluated, e.g., by gel staining, spectrophotometry, or terminus labeling etc.

“Nucleic acid polymer”, “polynucleotide” and “oligonucleotide” are used interchangeably and refer to a polymeric (2 or more monomers) form of nucleotides of any length, either ribonucleotides or deoxyribonucleotides. Although nucleotides are usually joined by phosphodiester linkages, the term also includes polymeric nucleotides containing neutral amide backbone linkages composed of aminoethyl glycine units. This term refers only to the primary structure of the molecule. Thus, this term includes double-and single-stranded DNA and RNA. It also includes known types of modifications, for example, labels, methylation, “caps”, substitution of one or more of the naturally occurring nucleotides with an analog, internucleotide modifications such as, for example, those with uncharged linkages (e.g., methyl phosphonates, phosphotriesters, phosphoamidates, carbamates, etc.), those containing pendant moieties, such as, for example, proteins (including for e.g., nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide. Polynucleotides include both sense and antisense strands. Genomic DNA, cDNA and mRNA are exemplary nucleic acid polymers.

The term “vector” is intended to include any physical or biochemical vehicle containing nucleic acid polymers of interest, by which those nucleic acid polymers are transferred into a host cell, thereby transforming that cell with the introduced nucleic acid polymers. Examples of vectors include DNA plasmids, viruses, and particle gun pellets.

The term “host cell” is intended to mean the target cell for vector transformation, in which the transferred nucleic acid polymer will be replicated and/or expressed.

As used herein “K2.254” refers to the monoclonal antibody produced by the cell line deposited with American Type Culture Collection (ATCC) 10801 University Blvd., Manassas Va. 20110-2209 USA on Dec.13, 2000 under the terms of the Budapest Treaty on the International Recognition of the Deposit of Microorganisms for the Purposes of Patent Procedure and having patent deposit designation PTA-2800.

### DETAILED DESCRIPTION

The following detailed description is provided to aid those skilled in the art in practicing the present invention. Even so, this detailed description should not be construed to unduly limit the present invention as modifications and variations in the embodiments discussed herein can be made by those of ordinary skill in the art without departing from the spirit or scope of the present inventive discovery.

All publications, patents, patent applications, databases and other references cited in this application are herein incorporated by reference in their entirety as if each individual publication, patent, patent application, database or other reference were specifically and individually indicated to be incorporated by reference.

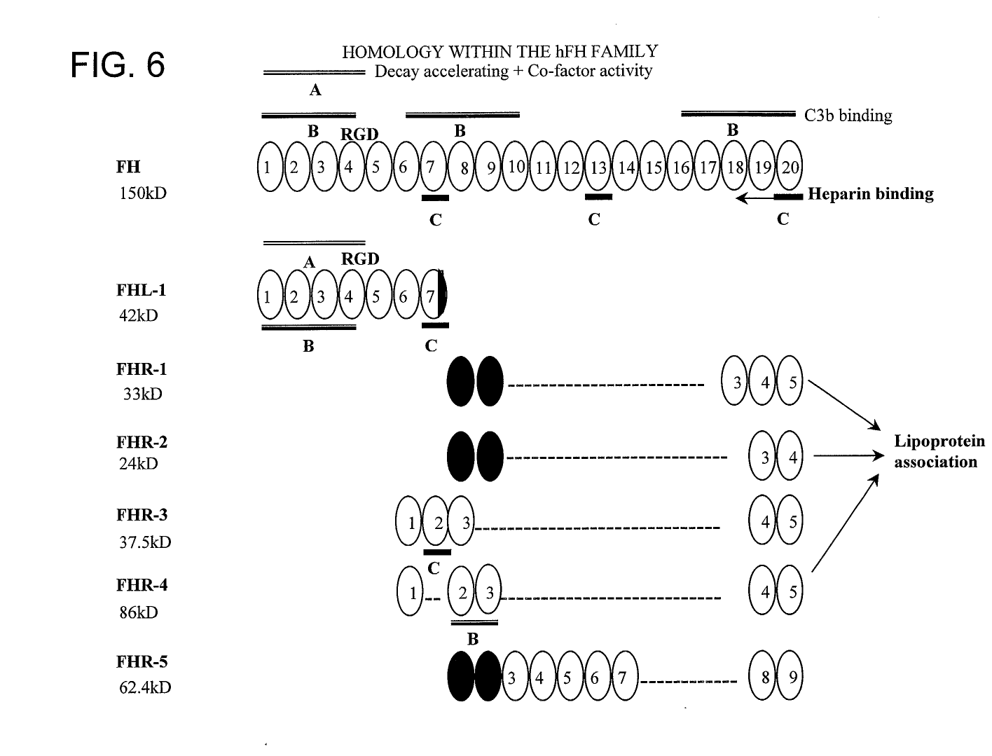

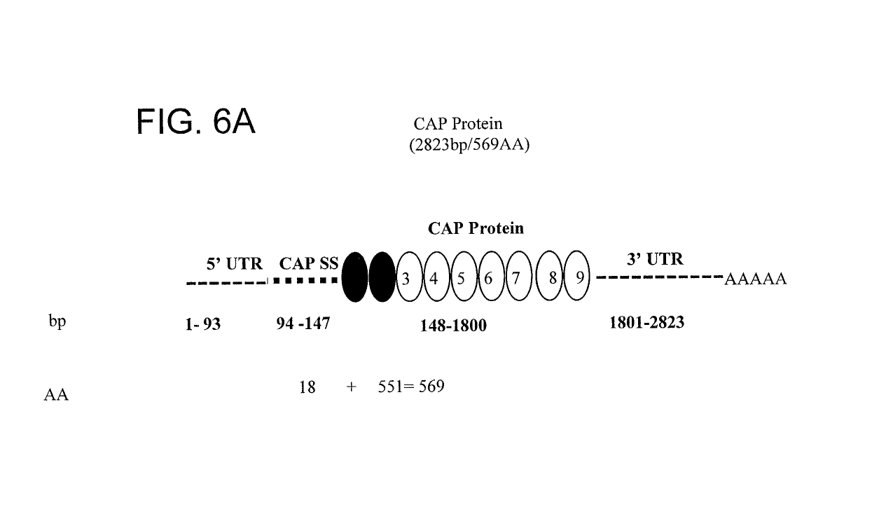

The novel human protein Factor H Related protein 5 (FHR-5) has been isolated, characterized, and recombinantly reproduced as described herein. FHR-5 has the primary amino acid sequence shown in SEQ ID NO. 2, and is encoded by the native cDNA sequence shown in SEQ ID NO. 1. Like other members of the FH family, FHR-5 is synthesized by the liver and is composed entirely of SCR domains. It can be found associated with mature complement C5b-9 complex, and is believed to play a role in the complement cascade. Without being bound to any particular theory, the homology of its SCRs to particular SCRs of other family members (FIG. 6) suggests that FHR-5 binds C3b and heparin. FHR-5 should also associate with lipoprotein as has been demonstrated for FHR-1 and FHR-2 (Park and Wright, J. Biol Chem., 271:18054-18060, 1996) and FHR-4 (Skerka, J. Biol. Chem., 272:5627-5634, 1997). Similarly, other complement regulatory proteins (C4-binding protein, CD59 and clusterin) have shown lipoprotein association (Valeva, Immunology, 82:28-33, 1994; Meri, Immunologist 2:149-155, 1994; Jenne, et al., J. Biol. Chem. 266:11030-11036, 1991; Jenne and Tschopp, Trends Biochem. Sci., 17:154-159, 1992; Funakoshi et al., Biochim. Biophys. Acta, 963:98-108, 1988). A role for the FHR proteins as constituents of lipoproteins has thus been suggested and could indicate a functional interaction of lipoproteins with the complement system.

FHR-5 is unique in the FH family in that it has been widely detected in vivo in association with the terminal C5b-9 complexes and appears to co-localize with these complexes in both normal and pathological human tissues. Thus, the FHR-5 protein is useful as a marker for the terminal C5b-9 complex. In a series of human renal biopsies, K2.254 appears to be a sensitive marker of complement activation. Despite this in vivo association with C5b-9, the similarity of the molecular structure of FHR-5 with other FH proteins would suggest a primary interaction in the complement system with the C3/C5 convertases.

Utilizing the primers listed in Table 2 and DNA amplification methods well known in the art, one of ordinary skill in the art may readily isolate the cDNA encoding FHR-5 from a liver cell cDNA library (such as the human liver Rapid-Scan™ Gene Expression Panel available from OriGene Technologies, Rockville, Md., USA) Such isolation is demonstrated in Example 2. Mature FHR-5 protein may then be produced in any recombinant expression system suitable for the production of eukaryotic proteins. Preferably, a eukaryotic expression system is used, such as the insect cell expression system utilized in Example 2. Utilization of animal cell systems is prudent to prevent improper glycosylation of the protein.

The state of the art of molecular biology is now sufficiently advanced that minor alterations can be made to a DNA sequence with relative ease and precision. A moderately skilled laboratory technician can follow the directions of one of the commercially available site-directed mutagenesis kits (for instance, the GeneEditor™ offered by Promega Corp., Madison, Wis. , USA) to effect any number of changes to a DNA nucleotide sequence. Also well known are the general rules governing the genetic code, by which triplet nucleotide codons are translated into an amino acid sequence by standard biochemical processes. Thus, the applicant considers the group of DNA sequences denoted by the consensus sequence of SEQ. ID NO. 3, which code for the amino acid sequence of FHR-5 in SEQ. ID. NO. 2, to be within the present invention. Although some variation in the genetic code such as codon usage and the GC% content occurs amongst some phyla, the rules governing these variations have also been well documented, and are within the reasonable skill of one versed in the molecular genetic arts. Thus, the applicant also considers any other nucleic acid sequence which encodes the amino acid sequence SEQ. ID NO. 2 to be within the scope of the present invention.

Although a particular embodiment of the nucleotide sequence disclosed herein is given in SEQ ID NO: 1, is should be understood that other biologically functional equivalent forms of the nucleic acid sequence of the present invention can be readily isolated using conventional DNA-DNA and DNA-RNA hybridization techniques. Thus the present invention also includes nucleotide sequences that hybridize to SEQ ID NO: 1 or its complement under high stringency conditions and encode proteins exhibiting the same or similar biological activity as that of protein of SEQ ID NO: 2 disclosed herein. In one embodiment, such nucleotide sequences hybridize to the nucleic acid of SEQ ID NO: 1 or its complement under high stringency conditions.

As is well known in the art, stringency is related to the Tₘ of the hybrid formed. The Tₘ (melting temperature) of a nucleic acid hybrid is the temperature at which 50% of the bases are base-paired. For example, if one the partners in a hybrid is a short oligonucleotide of approximately 20 bases, 50% of the duplexes are typically strand separated at the Tₘ. In this case, the Tₘ reflects a time-independent equilibrium that depends on the concentration of oligonucleotide. In contrast, if both strands are longer, the Tₘ corresponds to a situation in which the strands are held together in structure possibly containing alternating duplex and denatured regions. In this case, the Tₘ reflects an intramolecular equilibrium that is independent of time and polynucleotide concentration.

As is also well known in the art, Tₘ is dependent on the composition of the polynucleotide (e.g. length, type of duplex, base composition, and extent of precise base pairing) and the composition of the solvent (e.g. salt concentration and the presence of denaturants such formamide). One equation for the calculation of Tₘ can be found in Sambrook et al. (Molecular Cloning, 2nd ed., Cold Spring Harbor Press, 1989) and is:

Tₘ=81.5°C.−16.6(log₁₀[Na⁺])=0.41(%G+C)−0.63(% formamide)−600/L)

Where L is the length of the hybrid in base pairs, the concentration of Na⁺ is in the range of 0.01M to 0.4M and the G+C content is in the range of 30% to 75%. Equations for hybrids involving RNA can be found in the same reference. Alternative equations can be found in Davis et al., Basic Methods in Molecular Biology, 2nd ed., Appleton and Lange, 1994, Sec 6-8.

Methods for hybridization and washing are well known in the art and can be found in standard references in molecular biology such as those cited herein. In general, hybridizations are usually carried out in solutions of high ionic strength (6×SSC or 6×SSPE) at a temperature 20-25° C. below the Tₘ. High stringency wash conditions are often determined empirically in preliminary experiments, but usually involve a combination of salt and temperature that is approximately 12-20° C. below the Tₘ. One example of high stringency wash conditions is 1×SSC at 60° C. Another example of high stringency wash conditions is 0.1×SSPE, 0.1% SDS at 42° C. (Meinkoth and Wahl, Anal. Biochem., 138:267-284, 1984). An example of even higher stringency wash conditions is 0.1× SSPE, 0.1% SDS at 50-65° C. Exemplary conditions include initial hybridization in 5× SSPE,l- 5× Denhardt's solution, 100-200 μg/ml denatured heterologous DNA, 0.5% SDS, at 68° C. for a time sufficient to permit hybridization, e.g. several hours to overnight, followed by two washes in 2×SSPE, 0.1% SDS at room temperature and two additional 15 minute washes in 0.1×SSPE, 0.1% SDS at 42° C., followed by detection of the hybridization products. As is well recognized in the art, various combinations of factors can result in conditions of substantially equivalent stringency. Such equivalent conditions are within the scope of the present invention.

In addition to FHR-5, other substantially identical proteins may facilely be devised and synthesized by one of ordinary skill in the art. Although the understanding of the field of protein biochemistry is not as complete as that of molecular genetics, the person of ordinary skill in the art of biochemistry is capable of predicting, with reasonable certainty, when certain substitutions to the primary amino acid sequence structure of a protein will not result in any appreciable modification of a protein's structure or function. Such conservative substitutions are made by replacing an amino acid in the sequence with another containing a side chain with like charge, size, and other characteristics. For instance, the amino acid alanine, which has a small nonpolar methyl side chain, usually can be replaced by glycine, an amino acid which has a small nonpolar hydrogen side chain, without any noticeable effects. Because it is the interactive capacity and nature of a polypeptide that defines that polypeptide's biological functional activity, certain amino acid sequence substitutions can be made in a polypeptide sequence and nevertheless obtain a polypeptide with like properties.

In making such changes, the hydropathic index of amino acids can be considered. The importance of the hydropathic amino acid index in conferring interactive biologic function on a polypeptide is generally understood in the art (Kyte & Doolittle, J. Mol. Biol., 157: 105-132, 1982). It is known that certain amino acids can be substituted for other amino acids having a similar hydropathic index or score and still result in a polypeptide with similar biological activity. Each amino acid has been assigned a hydropathic index on the basis of its hydrophobicity and charge characteristics. Those indices are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is believed that the relative hydropathic character of the amino acid determines the secondary structure of the resultant polypeptide, which in turn defines the interaction of the polypeptide with other molecules, such as enzymes, substrates, receptors, antibodies, antigens, and the like. It is known in the art that an amino acid can be substituted by another amino acid having a similar hydropathic index and still obtain a functionally equivalent polypeptide. In such changes, the substitution of amino acids whose hydropathic indices are within ±2 is preferred, those which are within ±1 are particularly preferred, and those within ±0.5 are even more particularly preferred.

Substitution of like amino acids can also be made on the basis of hydrophilicity, particularly where the biological functional equivalent polypeptide or peptide thereby created is intended for use in immunological embodiments. U.S. Pat. No. 4,554,101, incorporated herein by reference, states that the greatest local average hydrophilicity of a polypeptide, as governed by the hydrophilicity of its adjacent amino acids, correlates with its immunogenicity and antigenicity, i.e. with a biological property of the polypeptide.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); prolin (−0.5±1); threonine (−0.4); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent polypeptide. In such changes, the substitution of amino acids whose hydrophilicity values are within ±2 is preferred, those which are within ±1 are particularly preferred, and those within ±0.5 are even more particularly preferred.

As outlined above, conservative amino acid substitutions are generally therefore based on the relative similarity of the amino acid side-chain substituents, for example, their hydrophobicity, hydrophilicity, charge, size, and the like. Exemplary substitutions which take various of the foregoing characteristics into consideration are well known to those of skill in the art and include: arginine and lysine; glutamate and aspartate; serine and threonine; glutamine and asparagine; and valine, leucine and isoleucine (See Table 1, below). The present invention thus contemplates functional or biological equivalents of FHR-5 which are substantially identical to FHR-5. These equivalent proteins are capable of associating with activated complement, and have at least 90% sequence identity to FHR-5 (or about 495 matching amino acids), as measured by the number of identical amino acids in the sequences when the sequence of the protein is aligned with SEQ ID NO. 2, and gaps are introduced in order to produce the greatest number of amino acid matches. More preferably such proteins have at least 95% sequence identity to FHR-5 (or about 523 matching amino acids), more preferably such proteins have at least 98% sequence identity to FHR-5 (or about 539 matching amino acids), more preferably such proteins have at least 99% sequence identity to FHR-5 (or about 545 matching amino acids), and most preferably such proteins have at least 99.5% sequence identity to FHR-5 (or about 548 matching amino acids). These equivalent proteins may be produced from the native cDNA sequence set forth in SEQ ID NO. 1 by the various methods of site-directed mutagenesis set forth above.

“Identity”, as is well understood in the art, is a relationship between two or more polypeptide sequences or two or more polynucleotide sequences, as determined by comparing the sequences. In the art, “identity” also means the degree of sequence relatedness between polypeptide or polynucleotide sequences, as determined by the match between strings of such sequences. “Identity” can be readily calculated by known methods including, but not limited to, those described in Computational Molecular Biology, Lesk, A. M., ed., Oxford University Press, New York (1988); Biocomputing: Informatics and Genome Projects, Smith, D. W., ed., Academic Press, New York, 1993; Computer Analysis of Sequence Data, Part I, Griffin, A. M. and Griffin, H. G., eds., Humana Press, New Jersey (1994); Sequence Analysis in Molecular Biology, von Heinje, G., Academic Press (1987); Sequence Analysis Primer, Gribskov, M. and Devereux, J., eds., Stockton Press, New York (1991); and Carillo, H., and Lipman, D., SIAM J. Applied Math, 48:1073 (1988). Methods to determine identity are designed to give the largest match between the sequences tested. Moreover, methods to determine identity are codified in publicly available programs. Computer programs which can be used to determine identity between two sequences include, but are not limited to, GCG (Devereux, J., et al., Nucleic Acids Research 12(1):387 (1984); suite of five BLAST programs, three designed for nucleotide sequences queries (BLASTN, BLASTX, and TBLASTX) and two designed for protein sequence queries (BLASTP and TBLASTN) (Coulson, Trends in Biotechnology, 12: 76-80 (1994); Birren, et al., Genome Analysis, 1: 543-559 (1997)). The BLAST X program is publicly available from the National Center for Biotechnology Information (NCBI) and other sources (BLAST Manual, Altschul, S., et al., NCBI NLM NIH, Bethesda, Md. 20894; Altschul, S., et al., J. Mol. Biol., 215:403-410 (1990)). The well known Smith Waterman algorithm can also be used to determine identity.

TABLE 1

| Original Residue | Exemplary Substitutions |
|---|---|
| Ala | Gly; Ser |
| Arg | Lys |
| Asn | Gln; His |
| Asp | Glu |
| Cys | Ser |
| Gln | Asn |
| Glu | Asp |
| Gly | Ala |
| His | Asn; Gln |
| Ile | Leu; Val |
| Leu | Ile; Val |
| Lys | Arg |
| Met | Met; Leu; Tyr |
| Ser | Thr |
| Thr | Ser |
| Trp | Tyr |
| Tyr | Trp; Phe |
| Val | Ile; Leu |

The present invention also relates to fragments, analogs and derivatives of the FHR-5 protein. The terms “fragment,” “derivative” and “analog” as used herein mean a polypeptide that binds to C3b and is recognized by monoclonal antibody K2.254 when associated with activated complement. For example, an analog includes a proprotein which can be cleaved to produce an active mature protein.

As demonstrated by the use of K2.254 to isolate FHR-5 in Example 2, FHR-5 comprises at least one antigenic epitope which is displayed when FHR-5 is associated with activated complement, but not when FHR-5 is in normal human serum. Thus FHR-5 can serve as a marker for complement activation. The association of FHR-5 with lipoprotein may explain the inability of K2.254 to detect FHR-5 in normal human serum. If FHR-5 is incorporated into a lipoprotein complex, the K2.254 Mab binding site may be masked. When this protein is dissociated from other proteins, perhaps through activation of complement, the epitope is presumably exposed. As FHR-5 could not be detected in normal human serum with the K2.254 Mab, a poly-histidine tag was incorporated into recombinant FHR-5 to ensure detection of the secreted protein. Interestingly, when cell culture supernatant was analyzed by Western blot both the K2.254 Mab and the anti-histidine antibody detected a product of approximately 67 kDa. Although not bound by any particular theory, these data support the view that FHR-5 may be associated in normal human serum with another molecule not present in the culture media. Thus, although the epitope recognized by K2.254 is not exposed in normal human serum, it is exposed when FHR-5 is substantially isolated in solution. Therefore, substantially isolated FHR-5, or a substantially identical protein with the same epitope, can be used to generate antibodies which are useful for the identification of activated complement, but which do not cross-react with normal human serum.

Antibodies may be generated to FHR-5 or a substantially identical protein according to standard methods known to those of ordinary skill in the art. The antibodies may be polyclonal, monoclonal, recombinant, chimeric, single-chain and/or bispecific, etc. The invention also encompasses immunologically active antibody fragments, for example, the Fab and Fab′ fragments. Various procedures known in the art can be used for the production of polyclonal antibodies which recognize epitopes of the polypeptides of this invention. For the production of antibody, various host animals can be immunized by injection with the polypeptide, or fragment or derivative thereof, including but not limited to rabbits, mice, rats, poultry, etc. Various adjuvants may be used to increase the immunological response, depending on the host species, including but not limited to Freund's, mineral gels such as aluminum hydroxide (alum), surface active substances such as lysolecithin, pluronic polyols, polyanions, peptides, oil emulsions, keyhole limpet hemocyanins, dinitrophenol, and potentially useful human adjuvants such as Bacille Calmette-Guerin and Corynebacterium parvum.

For the preparation of monoclonal antibodies directed toward FHR-5 or a substantially identical protein, any technique which provides for the production of antibody molecules by continuous cell lines in culture may be used. For example, the hybridoma technique originally developed by Kohler and Milstein which is described in Nature, 256:495-497 (1975), the trioma technique, the human B-cell hybridoma technique described by Kozbor et al. in Immunol. Today, 4:72 (1983), and the EBV-hybridoma technique to produce monoclonal antibodies described by Cole et al. in Monoclonal Antibodies and Cancer Therapy, Alan R. Liss, pp 77-96 (1985), are all useful for preparation of monoclonal antibodies in accordance with this invention. Other techniques are discussed in Monoclonal Antibodies: A Manual of Techniques by Heddy Zola (1987).

In addition, “humanized” antibodies to FHR-5 and substantially identical proteins can be produced utilizing techniques known in the art. Through this process, antibodies of a non-human species which recognize an epitope of FHR-5 are converted to chimeric partly-human antibodies through chemical or molecular genetics techniques. The humanizing process is thoroughly discussed in U.S. Pat. No. 5,861,155, incorporated herein by reference.

Alternatively, recombinant antibodies can be produced by the phage display method. Methods for the production and selection of antibodies using phage display are well known in the art and can be found, for example in Vaughan et al., Nature Biotech. 16:535-539, 1998; Watkins and Ouwehand, Vox Sanguinis 78:72-79, 2000; and the references cited therein.

Antibody production by phage display involves the generation of combinatorial libraries of immunoglobulin variable heavy chain (VH) and variable light chain (VL) sequences. These sequences are inserted into phage genes encoding coat proteins so that the VH and VL sequences are expressed (displayed) on the coat of filamentous bacteriophage. Phage expressing VH and VL regions of interest are selected by an affinity selection process commonly referred to as panning.

VH and VL sequences are generated by isolating mRNA from antibody secreting B-cells and amplifying the mRNA by RT-PCR using primers to conserved regions of the immunoglobulin gene. mRNA can be obtained from B-cells obtained directly from an animal, preferably an animal immunized with the antigen of interest, or from hybridoma cells producing antibodies against the antigen of interest. Once obtained, the VH and VL cDNA can be recombined by sequential cloning of VH and VL sequences into the same vector (Huse et al., Science, 246:1275-1281, 1989), by combinatorial infection using the loxCre site-specific recombination system of bacteriophage P1 (Waterhouse et al., Nuc. Acids Res., 21:2265-2266, 1993), or by PCR assembly (Clackson et al., Nature, 352:624- 628, 1991; Marks et al., J. Molec. Biol., 222:581-597, 1991). Alternatively, synthetic repertories of variable region sequences can be used as described, for example, in Griffiths et al. (EMBO J, 13:3245-3260, 1994).

Once a phage display library has been constructed, phage displaying reactive antibodies are selected by panning. Typically, purified antigen is attached to a solid substrate such as a plastic surface or an affinity chromatography column. The antigen may be attached to the surface directly or through an intermediary such as the streptavidin/biotin system. Phages to be selected are incubated with the antigen and non-binding phage washed away. A single round of selection can enrich for specific phage by 20 to 1,000 fold. Typically, several rounds of selection are carried out to increase specificity and affinity. Once phage displaying antibodies of the desired characteristics are identified, they are grown in a suitable bacterial host, the DNA encoding the antibody isolated, and the DNA sequenced. The antibody sequence can then be inserted into a suitable host cell for expression. Methods for the large scale production of antibodies from prokayotic, lower eukaryotic and eukaryotic cells are well known in the art and can be found for example in Frenken et al., (Res. Immunol., 149:589-599, 1998) and the references cited therein.

Alternatively, chimeric antibodies, including humanized antibodies described above, can be used. Chimeric antibodies are those in which different regions of the immunoglobuin molecule are from different sources. Typically, chimeric antibodies comprise a mouse variable region and a constant region derived from humans. Production of chimeric antibodies has become routine in the art and does not require any in depth structural knowledge of the antibody-antigen interaction (Watkins and Ouwehand, Vox Sanguinis, 78:72-79, 2000). Another form of chimeric antibody can be produced by the process known as “CDR grafting” (Jones et al., Nature, 321:522-525, 1986). CDRs are apical loops between the anti-parallel b-pleated sheets of a structure known as the immunoglobulin fold. The b-pleated sheets form a framework to correctly orientate the CDRs for interaction with the antigen. In CDR grafting, murine CDRs of a specific antibody are grafted onto an appropriate b-pleated sheet framework.

Additionally, antibodies can be obtained from transgenic animals and in particular transgenic mice (Bruggemann and Taussig, Curr. Opin. Biotechnol., 8:455-458, 1997). In this method, the endogenous mouse immunoglobulin genes are inactivated and replaced with unrearranged immunoglobulin sequences from humans. Monoclonal antibodies are then produced from the transgenic mice using the methods described above.

Once antibodies to FHR-5 or substantially identical proteins have been made, they can be screened for certain desirable activities. One preferred activity of the antibodies of the present invention is the ability to selectively identify activated complement. Antibodies to FHR-5 or substantially identical proteins can be screened for this activity by first testing them for reactivity with activated complement, such as that produced on guinea pig erythrocytes in Example 2.3. The screening can be done, for example, by ELISA, or by western blot, as in Example 2.4. Monoclonal antibodies which are reactive with activated complement are then screened for cross-reactivity with other complement proteins (C3, C5, C6, C7, C8, and C9) and normal human serum by western blot or ELISA, as in Example 1.6. Those antibodies which are not cross-reactive with other complement components or with normal human serum can be linked to radionuclides, fluorophores or fluorochromes, enzymes, vitamins, steroids or other detectable moieties and used to identify activated complement in various biopsied tissues, or other biological isolates. Several immunohistochemical methods are known in the art in which these antibodies might be used for such identification, including ELISA and flourescent microscopy techniques. Further instruction concerning such techniques can be found in Immunochemistry in Practice, Alan Johnston and Robin Thorpe, eds., 1996, and Immunochemical Protocols, Margaret M. Manson, ed., 1992.

Another preferred activity of the antibodies of the present invention is the ability to inhibit the biochemical functions of FHR-5 necessary for it to associate with activated complement. It is well known in the art that because antibodies are rather large, bulky molecules, they tend to cause steric interference with the normal functioning of a biomolecule when bound to epitopes close to those domains of the biomolecule necessary for its proper biological function. Thus, antibodies to FHR-5 or substantially identical proteins can be produced which, when bound to the protein, will inhibit the biochemical functions necessary for it to associate with activated complement. Antibodies FHR-5 or substantially identical proteins with this particular activity can be identified by screening for inhibition of FHR-5 association with activated complement. The first step in the screening process is the determination of the concentration of FHR-5 in normal human serum by ELISA or another immunoassay technique, preferably using a polyclonal antibody produced as described above. After the concentration of FHR-5 in a normal human serum reagent lot is determined, a stoichiometrically significant amount of a candidate antibody is added to one serum sample from the lot. Another serum sample without antibody is used as a control. Complement-lysed guinea pig erythrocytes are produced using both serum samples according to the method set forth in Example 2.3. The lysed erythrocytes may then be probed by western blotting with K2.254, as described in Example 2.4. A decrease in the reactivity with K2.254 of the candidate sample produced guinea pig erythrocytes as compared to the control will indicate that less FHR-5 has associated with activated complement, and that the antibody has the desired activity.

The present invention also involves recombinant polynucleotides comprising the substantially purified sequence of the present invention along with other sequences. Such recombinant polynucleotides are commonly used as cloning or expression vectors although other uses are possible. A recombinant polynucleotide is one in which polynucleotide sequences of different organisms have been joined together to form a single unit. A cloning vector is a self-replicating DNA molecule that serves to transfer a DNA segment into a host cell. The three most common types of cloning vectors are bacterial plasmids, phages, and other viruses. An expression vector is a cloning vector designed so that a coding sequence inserted at a particular site will be transcribed and translated into a protein.

Both cloning and expression vectors contain nucleotide sequences that allow the vectors to replicate in one or more suitable host cells. In cloning vectors, this sequence is generally one that enables the vector to replicate independently of the host cell chromosomes, and also includes either origins of replication or autonomously replicating sequences. Various bacterial and viral origins of replication are well known to those skilled in the art and include, but are not limited to the pBR322 plasmid origin, the 2μ plasmid origin, and the SV40, polyoma, adenovirus, VSV and BPV viral origins.

The polynucleotide sequences of the present invention may be used to produce proteins by the use of recombinant expression vectors containing the sequences. Suitable expression vectors include chromosomal, non-chromosomal and synthetic DNA sequences, for example, SV 40 derivatives; bacterial plasmids; phage DNA; baculovirus; yeast plasmids; vectors derived from combinations of plasmids and phage DNA; and viral DNA such as vaccinia, adenovirus, fowl pox virus, and pseudorabies. In addition, any other vector that is replicable and viable in the host may be used.

The nucleotide sequences of interest may be inserted into the vector by a variety of methods. In the most common method the sequence is inserted into an appropriate restriction endonuclease site(s) using procedures commonly known to those skilled in the art and detailed in, for example, Sambrook et al., Molecular Cloning, A Laboratory Manual 2^{nd} Ed., Cold Spring Harbor Press, (1989) and Ausubel et al., Short Protocols in Molecular Biology, 2^{nd} Ed., John Wiley & Sons (1992).

In an expression vector, the sequence of interest is operably linked to a suitable expression control sequence or promoter recognized by the host cell to direct mRNA synthesis. Promoters are untranslated sequences located generally 100 to 1000 base pairs (bp) upstream from the start codon of a structural gene that regulate the transcription and translation of nucleic acid sequences under their control. Promoters are generally classified as either inducible or constitutive. Inducible promoters are promoters that initiate increased levels of transcription from DNA under their control in response to some change in the environment, e.g. the presence or absence of a nutrient or a change in temperature. Constitutive promoters, in contrast, maintain a relatively constant level of transcription. In addition, useful promoters can also confer appropriate cellular and temporal specificity. Such promoters include those that are developmentally-regulated or organelle-,tissue- or cell-specific.

A nucleic acid sequence is operably linked when it is placed into a functional relationship with another nucleic acid sequence. For example, DNA for a presequence or secretory leader is operatively linked to DNA for a polypeptide if it is expressed as a preprotein which participates in the secretion of the polypeptide; a promoter is operably linked to a coding sequence if it affects the transcription of the sequence; or a ribosome binding site is operably linked to a coding sequence if it is positioned so as to facilitate translation. Generally, operably linked sequences are contiguous and, in the case of a secretory leader, contiguous and in reading phase. Linking is achieved by ligation at restriction enzyme sites. If suitable restriction sites are not available, then synthetic oligonucleotide adapters or linkers can be used as is known to those skilled in the art. Sambrook et al., Molecular Cloning, A Laboratory Manual, 2^{nd} Ed., Cold Spring Harbor Press, (1989) and Ausubel et al., Short Protocols in Molecular Biology, 2^{nd} Ed., John Wiley & Sons (1992).

Common promoters used in expression vectors include, but are not limited to, LTR or SV40 promoter, the E. coli lac or trp promoters, and the phage lambda PL promoter. Other promoters known to control the expression of genes in prokaryotic or eukaryotic cells can be used and are known to those skilled in the art. Expression vectors may also contain a ribosome binding site for translation initiation, and a transcription terminator. The vector may also contain sequences useful for the amplification of gene expression.

Expression and cloning vectors can and usually do contain a selection gene or selection marker. Typically, this gene encodes a protein necessary for the survival or growth of the host cell transformed with the vector. Examples of suitable markers include dihydrofolate reductase (DHFR) or neomycin resistance for eukaryotic cells and tetracycline or ampicillin resistance for E. coli.

In addition, expression vectors can also contain marker sequences operatively linked to a nucleotide sequence for a protein that encode an additional protein used as a marker. The result is a hybrid or fusion protein comprising two linked and different proteins. The marker protein can provide, for example, an immunological or enzymatic marker for the recombinant protein produced by the expression vector. The end of the polynucleotide can also be modified by the addition of a sequence encoding an amino acid sequence useful for purification of the protein produced by affinity chromatography. Various methods have been devised for the addition of such affinity purification moieties to proteins. Representative examples can be found in U.S. Pat. Nos. 4,703,004, 4,782,137, 4,845,341, 5,935,824, and 5,594,115. Any method known in the art for the addition of nucleotide sequences encoding purification moieties can be used for example those contained in Innis et al., PCR Protocols, Academic Press (1990) and Sambrook et al., Molecular Cloning, 2^{nd} Ed., Cold Spring Harbor Laboratory Press (1989).

More particularly, the present invention includes recombinant constructs comprising the isolated polynucleotide sequences of the present invention. The constructs can include a vector, such as a plasmid or viral vector, into which the sequence of the present invention has been inserted, either in the forward or reverse orientation. The recombinant construct further comprises regulatory sequences, including for example, a promoter operatively linked to the sequence. Large numbers of suitable vectors and promoters are known to those skilled in the art and are commercially available.

The polynucleotide sequences of the present invention can also be part of an expression cassette that at a minimum comprises, operably linked in the 5′ to 3′ direction, a promoter, a polynucleotide of the present invention, and a transcription termination signal sequence functional in a host cell. The promoter can be of any of the types discussed herein, for example, a tissue specific promoter, a developmentally regulated promoter, an organelle specific promoter, etc. The expression cassette can further comprise an operably linked targeting sequence, transit or secretion peptide coding region capable of directing transport of the protein produced. The expression cassette can also further comprise a nucleotide sequence encoding a selectable marker and a purification moiety.

A further embodiment of the present invention relates to transformed host cells containing the constructs comprising the polynucleotide sequences of the present invention. The host cell can be a higher eukaryotic cell, such as a mammalian cell, or a lower eukaryotic cell such as an insect cell or a yeast cell, or the host can be a prokaryotic cell such as a bacterial cell. Introduction of the construct into the host cell can be accomplished by a variety of methods including calcium phosphate transfection, DEAE-dextran mediated transfection, Polybrene mediated transfection, protoplast fusion, liposome mediated transfection, direct microinjection into the nuclei, biolistic (gene gun) devices, scrape loading, and electroporation.

### EXAMPLES

The following examples are intended to provide illustrations of the application of the present invention. The following examples are not intended to completely define or otherwise limit the scope of the invention.

### Example 1

### Production of Monoclonal Antibody K2.254

1.1 Isolation of Human Glomeruli and Preparation

The production of the K2.254 monoclonal antibody has been previously described (Murphy and d'Apice, Pathology, 20:130-136, 1988). Post mortem kidneys were obtained from 2 patients with glomerulonephritis who had died from causes other than end-stage renal failure. Portions of renal cortex were fixed in paraformaldehyde-lysine periodate (PLP) or mercuric formalin or fresh frozen for future immunohistological studies, and the remainder of the cortex was frozen for future mouse immunization. Four μm frozen sections of the fresh tissue were stained with fluorescein-labeled antibodies to human IgG, IgM, IgA and C3 and examined under an immunofluorescence microscope. Further sections of the mercuric formalin fixed tissue were processed for light microscopy.

Glomerular basement membranes (GBM) from each of the postmortem kidneys were diced and pressed through a 250 μm sieve, washed through a 180 μm sieve with cold 0.1 M phosphate-buffered saline (PBS) and glomeruli collected on a 106 μm sieve. The suspension of glomeruli was examined by phase contrast microscopy and, if there was more than 20% contamination with tubular fragments, passed through the sieves again. Glomeruli were washed twice in PBS at 4° C. and resuspended in PBS at a concentration of 12,500/ml.

Half of the suspension of glomeruli was then ultrasonicated in a Branson ultrasonicator using a microtip and a power output of 6. Three, 20 second bursts (with 1 min. cooling in ice water between each) were found to be sufficient for disruption of more than 90% of the glomeruli. Minimal ultrasonification was felt to be necessary as this procedure has been shown to damage components of the GBM and could conceivably detach or disrupt components of immune deposits attached to the GBM. Following ultrasonification the suspension was centrifuged at 1,500 g and washed twice in PBS.

1.2 Immunization of mice

The suspension of glomeruli was emulsified with a similar volume of Freund's complete adjuvant and 5 to 6 week-old Balb C mice were injected intradermally each with 0.8 ml of the emulsion (approx. 5,000 glomeruli). GBM suspensions were similarly emusified with an equal volume of Freund's complete adjuvant (such that 0.8 ml. of suspension contained the equivalent of 5,000 glomeruli) and injected intradermally into further mice. Twenty-one days later, the injection was repeated using the same emulsions in incomplete adjuvant. Each mouse received injections of renal tissue from the same kidney on both occasions. A further 14 days later, all mice received an intra peritoneal injection of the appropriate GBM preparation in PBS (containing the equivalent of approximately 3,000 glomeruli).

1.3 Determination of Anti HSA Antibodies in Immunized Mice

Prior to the intra peritoneal boost (3 days before fusions) all mice were eye bled. Serial dilutions of mouse sera were tested by radial immunodiffusion against carionic and native human serum albumin (HSA) in 1% agarose. Quantitative estimation of anti-HSA antibodies in mouse sera was also performed by enzyme linked immunoabsorbent assay (ELISA) as described in Example 1.6.

1.4 Fusion

Fusion techniques were adapted from the method of Oi and Herzenberg (Selected Methods in Cellular Immunology, Mishell and Shiigi, eds., Freeman, pp. 351-372, 1980). Three days after the intra peritoneal boost of GBM mice spleens were fused with 10⁷ SP2/0 myeloma cells in the presence of a 50% (w/v) solution of polyethylene glycol. Fusion mixtures were resuspended in HAT medium (RPMI 1640 with glutamine, penicillin, streptomycin, Na pyruvate, HEPES buffer and 15% fetal calf serum with added hypoxanthine, aminopterin and thymidine) and plated into three 96-well microtiter trays previously seeded with mouse thymocyte feeder cells. Supernatant from each well was sampled for testing by ELISA for the presence of mouse immunoglobulins.

1.5 Screening of Supernatants

All wells were tested by ELISA at 10 days for mouse immunoglobulin production. Wells producing mouse immunoglobulin were resampled 2 days later and tested by ELISA for activity against HSA. Hybridomas from wells with anti HSA activity were taken up into larger wells and cloned by limiting dilution in 96-well microtiter trays, using mouse thymocyte feeder cells. Positive wells were resampled 48 hours later and tested immunohistologically as follows:

Three μm frozen sections of PLP-fixed cortex from the kidney used for immunization and from the normal kidney were placed adjacent to one another on slides. Sections were incubated with hybridoma supernatants and then stained by a 4-layer immunoperoxidase technique. Hybridomas producing antibody reacting similarly in both normal kidney and the immunizing kidney were discarded. Those producing antibody apparently showing preferential reactivity with glomeruli immune deposits in the immunizing kidney were cloned.

1.6 Histological reactivity and characterization of “immune deposit reactive” monoclonal antibodies

Further sections of the immunizing kidney were examined by immunoperoxidase using each of the final cloned monoclonal antibodies and the pattern of glomerular and extraglomerular reactivity was noted.

Microtiter plates were preincubated at 4° C. with antigen in PBS, washed with PBS containing 0.5% Tween 20 (Bio Rad Laboratories, Hercules, Calif., USA) and sequentially incubated with MAb supernatant, alkaline phosphatase conjugated antibody to mouse IgG and IgM (Kirkegaard and Perry Laboratories, Gaithersburg, Md., USA) and substrate, p-nitrophenyl phosphate (Sigma Chemical, St. Louis, Mo., USA). Plates were read at 405 nm using a Titretek multiscan plate reader. Antigens used were purified human IgG, IgA and IgM, purified human complement components (C3, C5, C6, C7, C8, C9) and purified membrane attack complex of complement (MAC) extracted from lysed erythrocytes.

In ELISA studies where normal human serum was the antigen, the second antibody was horseradish peroxidase conjugated antibody to mouse immunoglobulins (DAKO, Denmark) and the substrate o-phenylene dismine. Plates were read at 485 nm.

Purified MAC and purified complement components were subjected to sodium dodecyl sulphate-polyacrylamide gel electrophoresis (SDS-PAGE). Protein was transferred to nitrocellulose using a BioRad transblot cell and strips of nitrocellulose were probed with monoclonal antibody supernatant followed by ¹²⁵I-labelled (Fab)₂ goat antimouse immunoglobulins (Pel-Freeze, Rogers, Ark., USA). Further strips were probed with antisera against human complement components C5, C6, C7, C8 and C9 followed by ¹²⁵I-labelled antisera to goat (Kirkegaard and Perry Laboratories) or rabbit (DAKO, Denmark) immunoglobulins. Nitrocellulose strips were then subjected to autoradiography.

From the monoclonal antibodies produced in this fashion, one candidate was found which reacted with glomeruli immune deposits, but did not react with purified human complement components (C3, C5, C6, C7, C8, C9), purified membrane attack complex of complement (MAC) extracted from lysed erythrocytes, or normal human serum. This antibody, K2.254, was used for further experimentation to determine the component of the glomerular immune deposits to which it had bound.

### Example 2

### Isolation of FHR-5 Protein and Elucidation of its Amino Acid Sequence

2.1 Materials

A rabbit polyclonal antibody against the surface of K562 cells was provided by Professor Paul Morgan (Cardiff, U.K.) and a monoclonal antibody against a neoantigen on polymerized human C9 (Wu-7-2) was provided by Dr Reinhardt Wurzner (Immunology, 74:132-138, 1991). A monoclonal antibody against human C9 (K2.322) and an isotype-matched control monoclonal antibody (K1.431) were produced as described above. FITC conjugated sheep anti-human albumin antibody was obtained from Silenus, Australia. The secondary antibodies used were FITC-conjugated rabbit anti-mouse immunoglobulins (DAKO, Carpinteria, Calif. USA), rabbit anti-mouse and donkey anti-goat immunoglobulins (Kirkegaard and Perry Laboratories) which were ¹²⁵I-labeled (Wurzner et al., Immunology, 74:132-138, 1991), horseradish peroxidase-conjugated rabbit anti-mouse immunoglobulins (DAKO), and sheep anti-Fluoroscein-POD Fab fragments (Roche Diagnostics, Mannheim, Germany)

A Tetra-His monoclonal antibody (Qiagen, Hilden, Germany) was used for detection of His-tagged recombinant protein expression.

2.2 Determination Of Mab K2.254 Reactivity with K562 Cells Exposed to Human Complement Attack

The human lymphoblast cell line K562 was obtained from American Type Culture Collection, Manassas, Va., U.S.A. Approximately one million K562 cells were incubated with 0.1 mg/ml anti-K562 antibody for 2 hours at 37° C., washed 3 times in PBS, and incubated with normal human serum or control heat-inactivated normal human serum, 1:2 dilution in PBS for 4 hours at 37° C. After incubation with sera, the cells were washed 3 times in PBS and the preparation divided, spun onto aminoalkylsilane (AAS) (Sigma) treated microscope slides, fixed in acetone for 10 minutes and air dried. The slides were incubated with K2.254, anti-C9 Mab K2.322, or isotype-matched control monoclonal antibody K1.431 (all antibodies at 10 mg/ml) for 1 hour at room temperature (RT) and washed 3 times in PBS. Slides were finally incubated in rabbit FITC-anti-mouse-mouse immunoglobulins (1:20) for 30 minutes at RT, washed a further 3 times and examined by epifluorescence microscopy.

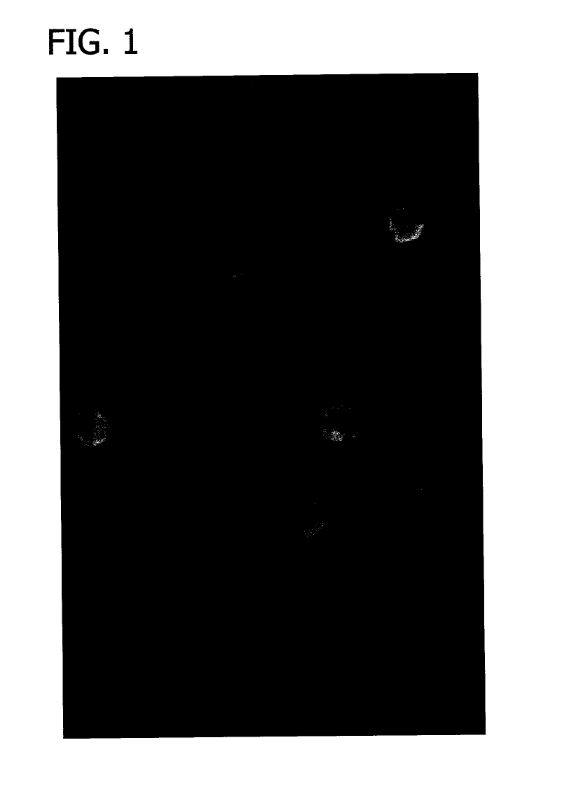

Mab K2.254 did not react with the surface of untreated K562 cells, K562 cells exposed to anti-K562 antibody and heat-inactivated human serum, or unsensitized K562 cells exposed to fresh human serum. After exposure to both anti-K562 antibody and fresh human serum, however, the cells showed morphological evidence of complement damage and strong cell surface staining for C9 (Mab K2.322) and K2.254 antigen (FIG. 1). When fresh human serum was substituted with guinea pig or rabbit serum there was morphological damage to the K562 cells but no cell surface reactivity with K2.254. These data suggest that the epitope for K2.254 is expressed on an antigen derived from human serum upon activation of complement.

2.3 Preparation of Complement-Lysed Human and Guinea Pig Erythrocyte Ghosts

Guinea pig erythrocytes (GPE) were directly lysed by reactive lysis using fresh normal human serum. Washed GPE were suspended 1:10 (v:v) in 0.9% sodium chloride solution and 5 ml of this preparation was incubated with 20 ml of normal human serum overnight at 37° C. The GPE ghosts were retrieved by centrifugation and washed 3-5 times in saline until the supernatant was clear.

Human erythrocytes (HE) (ABO blood group A) were antibody-sensitized by incubation (21° C. for 30 minutes) with 20% high titre human anti-blood group A serum. The HE were then washed once and incubated with normal human serum as described for GPE. To prepare negative control erythrocyte ghosts, 5 ml of the washed 1:10 GPE and HE suspensions were osmotically lysed by the addition of 10 ml distilled water and the erythrocyte ghosts recovered and washed as above.

2.4 Western Blotting

Normal human serum, SC5b-9 complexes purified from activated serum (Wurzner et al., Immunology, 74:132-138, 1991), erythrocyte ghosts and affinity-purified K2.254 antigen were prepared and electrophoresed on 7.5% or 4-16% gradient SDS-PAGE gels (Laemmli, Nature, 227:680-685, 1970), transferred to nitrocellulose (Trans-Blot®Transfer Medium, Bio-Rad, Hercules, Calif., USA) using a Bio-Rad transblot cell (Bumette, Anal Biochem., 112:195-203, 1981), blocked with 5% dried milk in PBS (phosphate buffered saline) for 1 hour at RT, then probed with appropriate monoclonal and polyclonal antibodies. Membranes were washed 5 times for 5minutes in PBS+0.05% Tween-20 followed by incubation with either ¹²⁵I labeled secondary antibody or rabbit anti-mouse-HRP (1:1000), 1 hour at RT. Following washing, radioactive bands were detected by autoradiography and HRP labeled bands using SuperSignalÒWest Pico Chemiluminescent Substrate (Pierce, Rockford, Ill., USA) as specified.

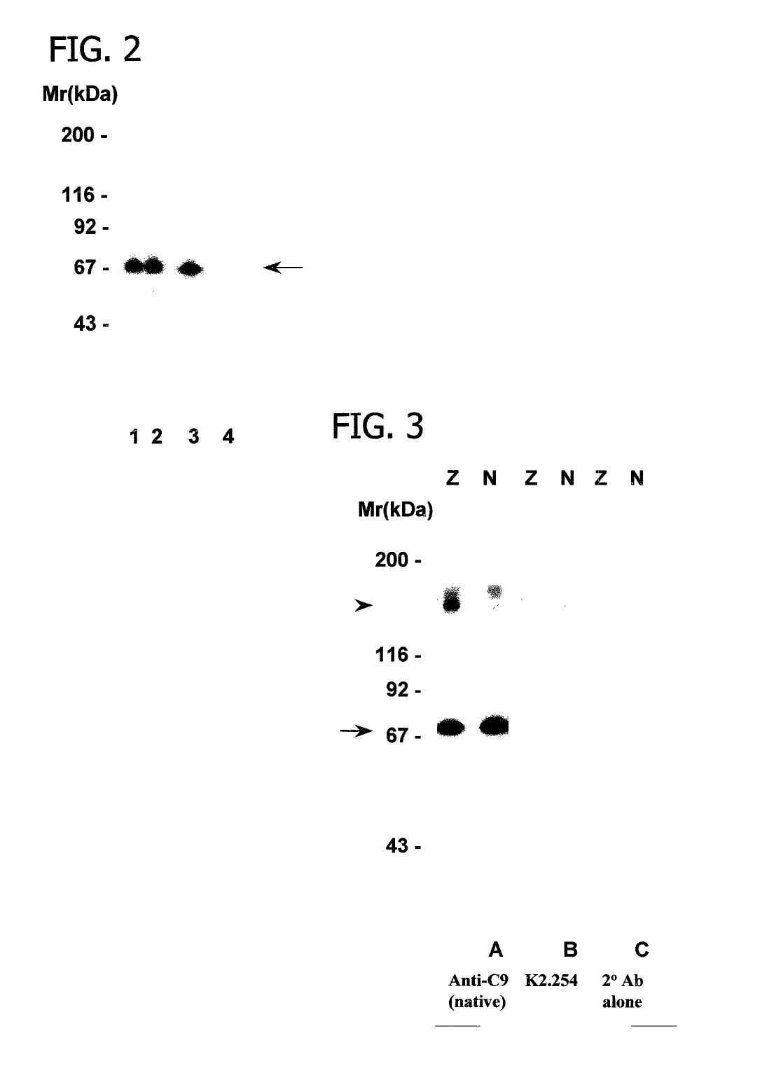

To further analyze the antigen recognized by K2.254, membranes from guinea pig or human erythrocytes lysed with human serum were examined by Western immunoblotting (FIG. 2). K2.254 identified a band of approximate 65kD in complement lysed HE and GPE membranes (lanes 1,2,3) but not in control water-lysed membranes (lane 4).

Normal human serum contained C9 (FIG. 3A) but did not react with Mab K2.254 (FIG. 3B). Following zymosan pre-treatment of the serum to activate complement, dimeric C9 was detected, indicating activation of complement, but again no reactivity with K2.254 was observed. Similarly, Western blotting of purified SC5b-9 failed to identify the antigen recognized by K2.254.

2.5 Affinity Purification of K2.254 Antigen

Complement-lysed GPE ghosts were extracted with 5 ml of 1% digitonin (Sigma) in 0.1M PBS overnight at 4° C. Affinity chromatography was performed using a 10 ml column of cyanogen bromide-activated Sepharose 4B (Amersham Biotech, Uppsala, Sweden) to which 50 mg of the K2.254 Mab had been coupled according to the manufacturers instructions. The 5 ml digitonin extract was passed over the column and collected. The column was then washed with 200 volumes of PBS containing 0.01% Triton X-100 (PBS/Triton) and eluted with 10 ml 0.05M diethylamine, pH 11.5. The sample was eluted into 1 ml of 1M Tris-HCl (pH 7.0). Each 5 ml extract was passaged twice through the column and the eluates from 3 samples (6 passages) were pooled, dialyzed extensively against PBS/Triton and then concentrated to 0.5 ml (YM30 membrane and Centricon 30; Amicon, Beverly, Mass., USA). The concentrated eluate was then subjected to SDS-PAGE and western blotting as described above.

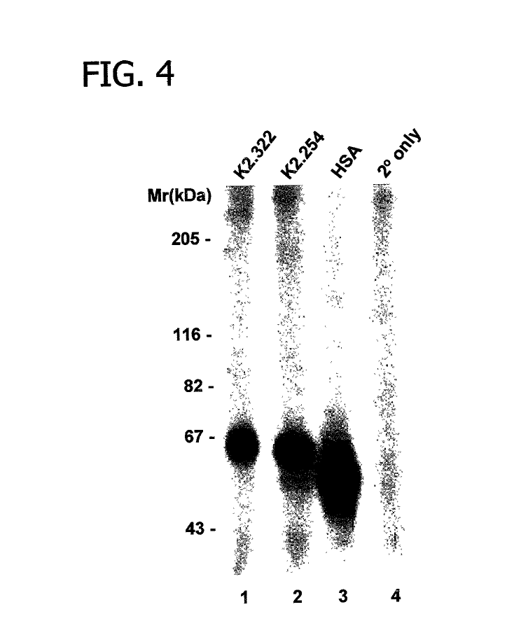

Due to the strong reactivity of K2.254 with human complement-lysed GPE, digitonin-extracted GPE ghosts were used to extract and affinity purify the associated protein using a K2.254 affinity column. The affinity-purified material reacted with K2.254 in Western blot but was contaminated with C9 and human serum albumin (HSA) (FIG. 4), necessitating further purification.

2.6 Reverse-Phase High Performance Liquid Chromatography (HPLC)

Separation from HSA and C9 was achieved by reverse-phase HPLC. Fractions containing material reactive with K2.254 but not human C9 or HSA were pooled, lyophilized and subsequently used for peptide sequencing. Samples of the concentrated affinity column eluate were precipitated with 10% trichloroacetic acid (TCA), washed with an 80% ether/20% ethanol solution and redissolved in 100 ml of 6M guanidinium hydrochloride, 0.1M Tris, pH 8.0 (HCl)+0.05% trifluoroacetic acid (TFA), and subjected to reverse-phase HPLC on a Pharmacia SMART HPLC system using a 2.1 mm ID× 250mm Brownlee RP300 7Angstrom C8 column. (Solvent A 0.1% TFA, Solvent B 60% acetonitrile 0.1 % TFA, flow 100 microliters per minute). A gradient of 0-100% B in 60 minutes was employed and peaks were monitored at 280nm and collected manually. Fractions were analyzed by Western blot to detect elution of individual components, and fractions containing K2.254 binding activity were pooled and lyophilized.

2.7 Peptide Sequence Analysis

Lyophilized fractions from the HPLC were reconstituted in SDS sample buffer and run on 10% SDS PAGE. Bands were excised, reduced, pyridylethylated and subjected to in-gel digestion with Achrombacter Lys-C endoproteinase (Wako) (Mitchelhill et al., J. Biol. Chem., 272:24475-24479, 1997). Extracted peptide mixtures were subject to HPLC on a Pharmacia SMART system using a 1 mm ID X 250 mm, SGE 300 micron, 5 angstrom, C18 GLT column. (Solvent A 0.1% TFA Solvent B 60% acetonitrile 0.075% TFA Flow 40 microliters per minute). A gradient 0-100% B in 150 minutes was run, peaks monitored at 214, 254 and 280 nm and individual peptides were manually collected. Peptide homogeneity and mass were established on a Perseptive Biosystems Voyager DE MALDI mass spectrometer and amino acid sequence was determined on a Hewlett Packard G1000A Protein Sequencer. Homology searches were performed using non-redundant protein databases (Genbank CDS translations+PDB+Spupdate+PIR) and 6 translations of the EST database.

After in-gel proteolysis and HPLC separation of peptides, the sequence of 13 purified peptides was obtained. All of these peptides showed some homology with members of the Factor H (FH) family of proteins. Table 3 shows the peptide sequences and lists the peptides to which they showed strongest homology.

2.8 RNA Preparation

Total RNA was extracted from a piece of normal, non-infiltrated human liver, obtained from one patient during liver resection. The tissue was homogenized and RNA isolated with TRIZOL reagent (Life Technologies, Grand Island, N.Y., USA) according to the manufacturer's instructions. Ten mg of total RNA was treated with DNase to remove contaminating genomic DNA and poly (A)⁺RNA was isolated using an Oligotex mRNA kit (Qiagen, Hilden, Germany).

2.9 cDNA Synthesis and Cloning

Double stranded cDNA was synthesized from 2 mg human liver poly(A)⁺RNA using either oligo(dT) or random primers and Superscript II RNase H reverse Transcriptase (Life Technologies) according to the manufacturer's instructions.

Gene-specific primers (GSP-1 and GSP-2, see Table 2) were use to amplify an initial product from human liver cDNA. Touchdown PCR, with annealing temperatures of 70° C. down to 65° C., was carried out in a DNA Engine thermal cycler (MJ Research, Watertown, Mass., USA) for 40 cycles. 3′ and 5′ RACE (rapid amplification of cDNA ends)(See, Innis et al., PCR Protocols, Academic Press, pp. 28-38, 1990) was then performed using GSPs, Advantage DNA polymerase and the Marathon cDNA Amplification kit (Clontech Laboratories, Palo Alto, Calif. USA ). Full length cDNA was amplified from human liver poly (A)⁺ RNA as above using the TCAP-F primer (Table 2) and the AP1 primer supplied with the Marathon Kit.

PCR products were gel-purified (Qiagen Gel Extraction Kit) and cloned into pGEM-T Easy (Promega, Madison, Wis. , USA). Oligonucleotide primers were synthesized by Life Technologies.

TABLE 2
Gene specific primers used in the cDNA
synthesis and cloning

| | | SEQ |
|---|---|---|
| | | ID |
| Primer | Sequence | NO |
| GSP-1: | 5′-GGTGTGTTGCAACACACATAGGAAGCTCT-3′ | 4 |
| GSP-2: | 5′-GTCATGTTGCCCATTTTAGAAGCCAATG-3′ | 5 |
| CAP-F1: | 5′-GGAGAAGGAACACTTTGTGA-3′ | 6 |
| CAP-F2: | 5′-ATAAGAGTTGGATCAGACTC-3′ | 7 |
| CAP-F3: | 5′-GTATATCCTCCAGGGTCAAC-3′ | 8 |
| CAP-F4: | 5′-GTGGATACATACCTGAACTCG-3′ | 9 |
| CAP-F5: | 5′-TCACCAACACCGAAGTGTCTC-3′ | 10 |
| CAP-F6: | 5′-CCATCTACGCTAAAAGTAGCC-3′ | 11 |
| CAP-R1: | 5′-GAACATAACCGTACTCGAG-3′ | 12 |
| CAP-R2: | 5′-CCACATGATCGTACTTGTC-3′ | 13 |
| CAP-R3: | 5′-GAGCATCTACATTGGCTTC-3′ | 14 |
| CAP-R4: | 5′-GTTGACCCTGGAGGATATAC-3′ | 15 |
| CAP-R5: | 5′-CTGAGCATTAGGTATCTGAGG-3′ | 16 |
| CAP-R6: | 5′-CTTCCCCTGTACGAACTTGGGAA-3′ | 17 |
| 707-F2: | 5′-GTTCAGACATCTTCAGATACAGGC-3′ | 18 |
| 707-F3: | 5′-CAGTTAAAATGGAGAAACGATGG-3′ | 19 |
| 3′BAC: | 5′-GGACTAGTGGCTACTTTTAGCGTAGATGG-3′ | 20 |
| 5′BAC(C): | 5′-GAAGGAACACTTTGTGATTTTCC-3′ | 21 |
| TCAP-F: | 5′-CGGGCAGGTGCTTGTTACTGTTAATG-3′ | 22 |
| TCAP-R: | 5′-GCTACAACCACAAAAGTTGACAGCT-3′ | 23 |
| Mlu 1-F: | 5′-CGACGCGTGAAGGAACACTTTGTGA-3′ | 24 |
| Sal 1-R: | 5′-GCCGTCGACCCTTCACATATAGGATATTC-3′ | 25 |

2.10 DNA Preparation and Sequencing

Plasmid DNA was prepared from bacterial cultures using Qiagen Plasmid Mini Kits. Correct identity of the cloned fragments was confirmed by sequencing, PCR, and restriction enzyme analysis. DNA sequencing reactions were performed using BIGDYE Terminator Cycle Sequencing Ready Reaction (PE Applied Biosystems, Foster City, Calif. USA) and electrophoresed by The Australian Genome Research Facility, Melbourne, Australia. Both strands of cDNA clones were sequenced using standard vector primers and several of the internal primers listed in Table 2.

Alignment of the peptides with human FH was used to design primers for cDNA isolation. Human liver was chosen as the source of RNA because members of the FH gene family are expressed in liver. Two degenerate primers, 254-F1 (5′-GGNGARTGYCAYGTNCCNAT, SEQ ID NO: 26) and 254-R1 (5′-CARTCNACYTCNGGRTTCCA, SEQ ID NO: 27), were used to amplify a 709 bp product. Sequencing of this PCR product revealed an open reading frame (ORF) encoding a partial protein sequence with 55.5% identity to amino acids 549 to 785 of human FH. The deduced protein sequence contained perfect matches with peptides H 1113, H1114, H1116 and H1117 (Table 3). Because the new protein was similar but not identical to FH, it was designated Factor H-Related Protein 5 (FHR-5).

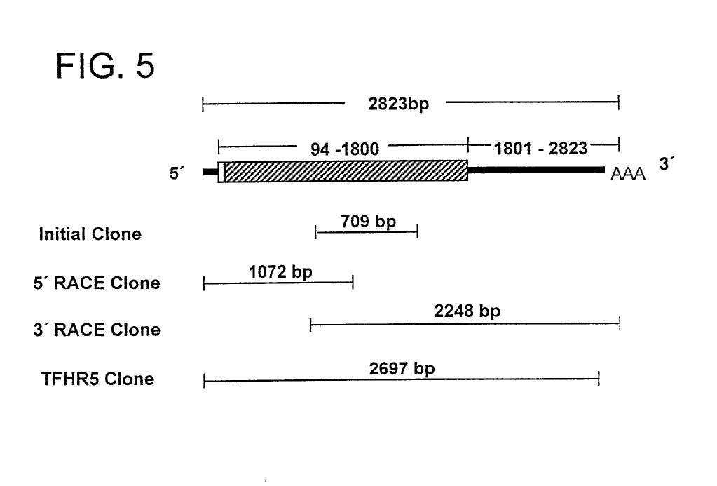

3′ and 5′ RACE was then performed yielding 1072bp and 2248bp products, respectively. Sequencing of these clones enabled the amplification of a single full length FHR-5 cDNA from liver RNA. FIG. 5 shows the position and overlap of these clones to produce the 2823 bp cDNA. To minimize the possibility of PCR-generated errors, the full sequence was obtained from clones generated from at least two identical PCRs.

2.11 Northern Blot Analysis

Ten mg of total RNA or 1 mg poly (A)⁺ RNA was electrophoresed on a 1% agarose gel containing formaldehyde and transferred to nylon membrane GeneScreen Plus (NENÔ-Life Science Products, Boston, Mass. USA) by standard procedures (Sambrook et al., Molecular Cloning, 2nd Ed., Cold Spring Harbor Laboratory Press, 1989). RNA was crosslinked to the membranes using a UV Stratalinker^{O} 1800 (Stratagene, La Jolla, Calif. USA) and blots were pre-hybridized in Rapid-hyb buffer (Amersham Biotech, Uppsala, Sweden) at 65° C. for 1 hour. Hybridization with specific probes labeled with ³²P-a using the Megaprime DNA labeling system (Amersham Biotech), was performed at 65° C. for 2 hours. Membranes were washed for 15 minutes in 2×SSC+0.1% SDS at 65° C., 1×SSC+0.1% SDS at 65° C. and 0.1×SSC+0.1% SDS at RT and filters exposed at −70° C. overnight.

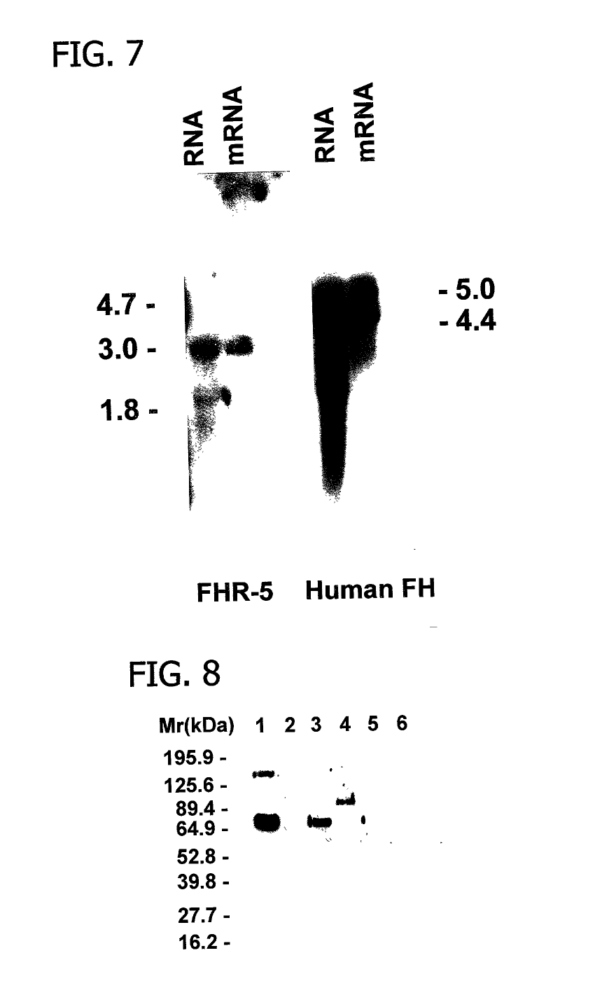

A Northern blot of human liver RNA was probed with the 709 bp FHR-5 PCR fragment and a human Factor H 711 bp PCR fragment amplified from the same region. The FHR-5 fragment hybridized to a single mRNA species with an estimated size of 3.0 kilobases. (FIG. 7) There was no cross-reactivity with any other mRNA species. When the same blot was probed with the FH probe, two mRNA species were detected at 4.4 and 5.0 kilobases. Two such species have been previously described. The FH probe did not cross-hybridize with the FHR-5 mRNA species, further confirming the identification of a specific FHR-5 mRNA.

2.12 Construction of Plasmids for Baculovirus Expression

The complete cDNA sequence is shown in SEQ ID NO. 1, and the deduced amino acid sequence is shown in SEQ ID NO. 2. There was an ORF from bases 94 to 1800, with the region of the start codon displaying good agreement with the Kozak consensus sequence for translation initiation. The ORF encoded an 18-amino acid signal sequence and 551 amino acids of the mature protein. The ORF was followed by 1000 bp of 3′ untranslated sequence including a consensus polyadenylation signal at position 2705-2710 and a poly A⁺ tail.

Mature FHR-5 has a predicted molecular weight (nonglycosylated) of ˜62,650 Da, and two potential N-linked glycosylation sites at positions 108 and 382, see SEQ ID NO. 2.

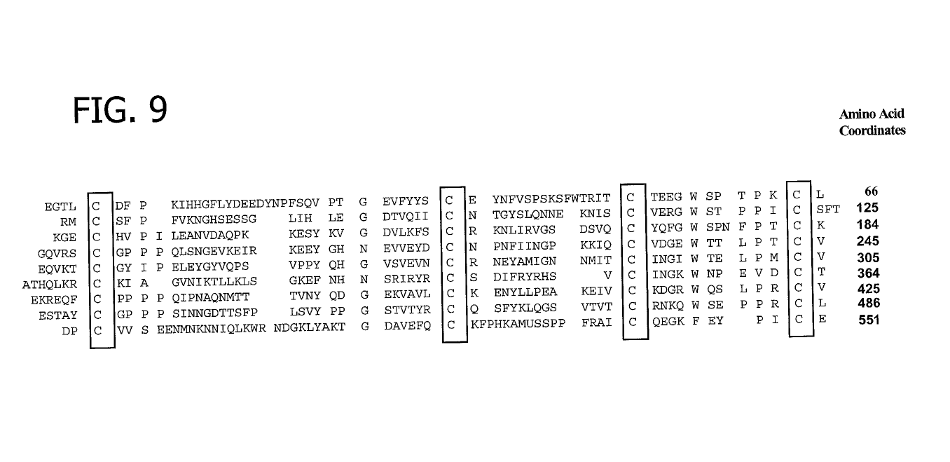

The secreted form of FHR-5 contains 9 short consensus repeat (SCR) domains. Each SCR contains the four characteristic cysteine (C) residues (boxed in FIG. 9) and additional conserved amino acids (aligned in FIG. 9). All of the peptide fragments isolated could be assigned to the mature protein.

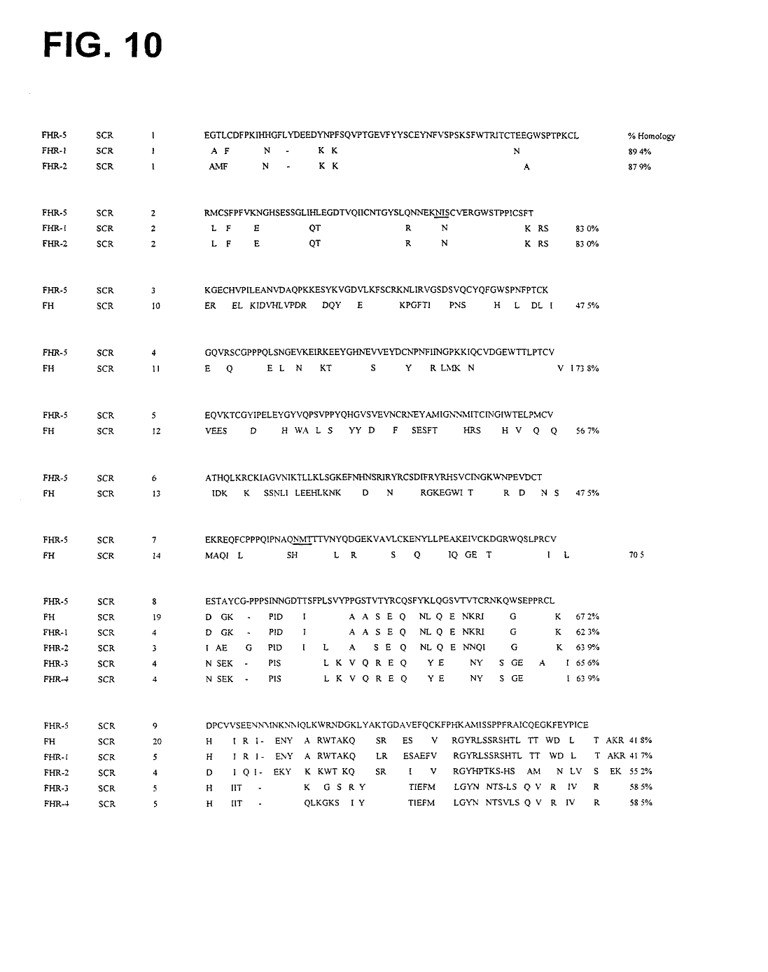

SCRs of FHR-5 display varying homology to members of the FH family (FIG. 10). SCRs 1 and 2 of FHR-5 are homologous to SCRs 1 and 2 of FHR-1 and FHR-2, SCRs 3-7 display homology ranging to SCRs 10-14 of FH and SCRs 8 and 9 display homology to last two SCRs of FH and all FHR proteins. A schematic representation of the SCR alignment is shown in FIG. 6.

A 1,672 bp fragment was amplified from the full length (3.0 Kb) cDNA by PCR (touchdown 56° C.-50° C.) using Pfu DNA polymerase (Roche Diagnostics, Mannheim, Germany). The forward primer (Mlu1-F, see Table 2) was designed to anneal immediately downstream of the signal peptide-encoding region and contained an Mlu1 restriction site at the 5′ end. The reverse primer (Sal1-R, see Table 2) annealed immediately downstream of the open reading frame (ORF) and incorporated a Sal1 restriction site at the 5′ end. The PCR product was digested with Mlu1/Sal1 (Promega, Madison, Wis., USA) and cloned into the pFASTBAC1-His¹⁰⁺ vector which was partially digested with Mlu1, gel-purified then treated with Xhol. pFASTBAC1-His¹⁰⁺, a modified version of pFASTBAC1 (Life Technologies) was kindly provided by Dr Brett Cromer of SVIMR, encodes a gp67 signal peptide to ensure efficient secretion and a 10-histidine N-terminal epitope tag for detection and purification. The complete sequences of the insert and its junctions were confirmed by sequencing.

2.13 Recombinant Expression and Purification of FHR-5 in Insect Cells

A modification of the Bac-to-Bac Baculovirus Expression Systems protocol (Life Technologies) was used to obtain purified recombinant baculovirus. Recombinant pFastBac1-His¹⁰⁺ vector was transferred into DH10Bac-competent cells and cultured overnight in 4 ml S.O.C medium containing gentamicin, tetracycline and kanamycin. In order to obtain a pure recombinant virus strain without the need for plaque assay analysis, recombinant Bacmid DNA was isolated as described and transfected into DH10 competent cells (Life Technologies) by electroporation (2.5 kvolts, 25 mFD, 200 ohms). Transfected cells were cultured on Luria Agar plates containing 5-bromo-4-chloro-3-indoyl b-D-galactoside (X-Gal), isopropyl b-D-thiogalactoside (IPTG), kanamycin and gentamicin for 24 hours. Colonies containing pure recombinant Bacmid DNA were selected by blue/white screening, and total DNA was prepared and used to transfect Sf9 cells.

Spodopterafrugiperda cells (Sf9) were maintained at 27° C. in Grace's insect medium (Invitrogen, Carlsbad, Calif., USA) supplemented with 10% fetal bovine serum (FCS, Life Technologies). Cells were grown either as suspension cultures in spinner flasks or as monolayer cultures in tissue culture flasks. Prior to transfection with recombinant expression plasmids, cells were adapted to serum-free conditions using HyQÒSFX-Insect Serum-Free Cell Culture Medium (Progen, Ipswich, Qld, Australia).

Sf9 cells were seeded at 1 times 10⁶ per 35-mm well and transfected using the Bac-to-Bac Baculovirus Expression System according to the manufacturer's procedures. Culture supernatant was collected after 3-4 days and analyzed for synthesis of recombinant proteins by Western Blot. Fifteen ml of insect cell culture medium was added to 5 ml of 4× non-reducing or 4× reducing sample buffer and 20 ml samples were electrophoresed on 7.5% SDS-PAGE gels and transferred to nitrocellulose as previously described. Blots were probed with K2.254 or anti-Tetra-His Mabs (1:1000) (Qiagen), and anti-human Factor H Mab (1:1000) (Serotec, U.K) as a negative control. The secondary antibody was rabbit anti-mouse-HRP (DAKO).

Recombinant virus was isolated from culture medium 3 days post transfection and viral stocks were established. For purification of recombinant protein, 1×10⁶ Sf9/High Five cells in 40 ml HyQ medium were infected with purified recombinant virus. Four days after infection, recombinant protein was purified using Ni²⁺- NTA agarose (Qiagen) chromatography as previously described (Kuhn and Zipfel, Euro. J. Immunol., 26:2383- 2387, 1995) and analyzed by Western blot (FIG. 8).

A recombinant version of FHR-5 incorporating a polyhistidine epitope tag at the C-terminus was expressed in insect cells using a baculovirus expression system. Western blotting with the Tetra-His antibody (FIG. 8, lane 3) detected a 65 kD molecule in the cell culture medium of infected insect cells. Upon reduction a band of ˜90 kD was seen (FIG. 8, lane 4) confirming the protein produced contains internal disulfide bonds, characteristic of SCR-containing molecules. The K2.254 antibody was also used in Western blotting and detected an identical band in the non-reduced sample (FIG. 8, lanel). A higher protein band was also detected at an Mr of ˜40 kDa probably indicating a dimer formation although this was not detected with the anti-His antibody. No band was seen in the reduced sample probed with K2.254, indicating a loss of the epitope after reduction of disulfide bonds.

TABLE 3
Amino acid homology
of HPLC-purified peptides to the FH and FHR proteins.

| PEP- | | | |
|---|---|---|---|
| TIDE | | HOMOLOGY | |
| # | SEQUENCE | COORDINIATES | |
| H1113 | WNPEVDCTEK | Factor H | 416-423 |
| H1114 | GECHVPILEANVDAQPK | Factor H | 549-565 |
| H1115 | FEYPICE | Factor H | 1205-1211 |
| H1116 | EEYGHNEVVEYDCNPNFIING- | Factor H | 629-651 |
| | PK | | |
| H1117 | IQCVDGEWTTLPTCV | Factor H | 653-672 |
| H1118 | GWSTP | FHR1/2 | 115-119 |
| H1119 | SFWTRITCTEEG | FHR-2 | 47-58 |
| H1120 | MCSFP | unassigned | (SCR 2 |
| | | | FHR-5) |
| H1121 | AMISSPPFRAICQEGK | unassigned | (SCR20 |
| | | | FHR-5) |
| H1122 | TGDAVEFQCK | Factor H | 1175-1184 |
| H1123 | DGRWQSLPR | Factor H | 837-845 |
| H1124 | VALVCK | Factor H | 817-822 |
| H1125 | ENYLLPEAK | Factor H | 823-831 |

The amino acid coordinates of the region of protein with the strongest homology to the relevant peptide are shown in the third column. The majority of the peptides displayed high homology with human FH while peptides H1118 and H1119 were most closely related to the FHR-1 and FHR-2. Peptides H1120 and H1121 showed no significant homology to the FH family of proteins but were later assigned to the FHR-5 protein.

### Example 3

### In Vitro Binding Studies with Recombinant FHR-5

Recombinant FHR-5 or Factor H (Sigma) were serially diluted 1:2 in 0.1 M carbonate buffer, pH 9.3. Duplicate wells in microtitre plates were incubated with dilutions of FHR-5 or Factor H from 50 pmol/well to 0.4 pmol/well. The plates were blocked and wells incubated with human C3b, C5b-6 or HSA at 10 μg/well. C3b was detected by incubation with polyclonal rabbit anti-C3 antibody (1:1000 dilution) followed by peroxidase-conjugated swine anti-rabbit antibody (1:2000 dilution). C5b-6 and HSA were detected using K1.115 (anti-C6) and anti-HSA monoclonal antibodies, respectively, (1:500 dilution) followed by peroxidase-conjugated rabbit anti-mouse antibody (1:2000 dilution). The ELISAs were developed with O-phenylenediamine dihydrochloride. The reaction was stopped with 4M H₂SO₄ and the optical density of the solution was measured at a wavelength of 492 nm using a Behring EL31 microplate reader.

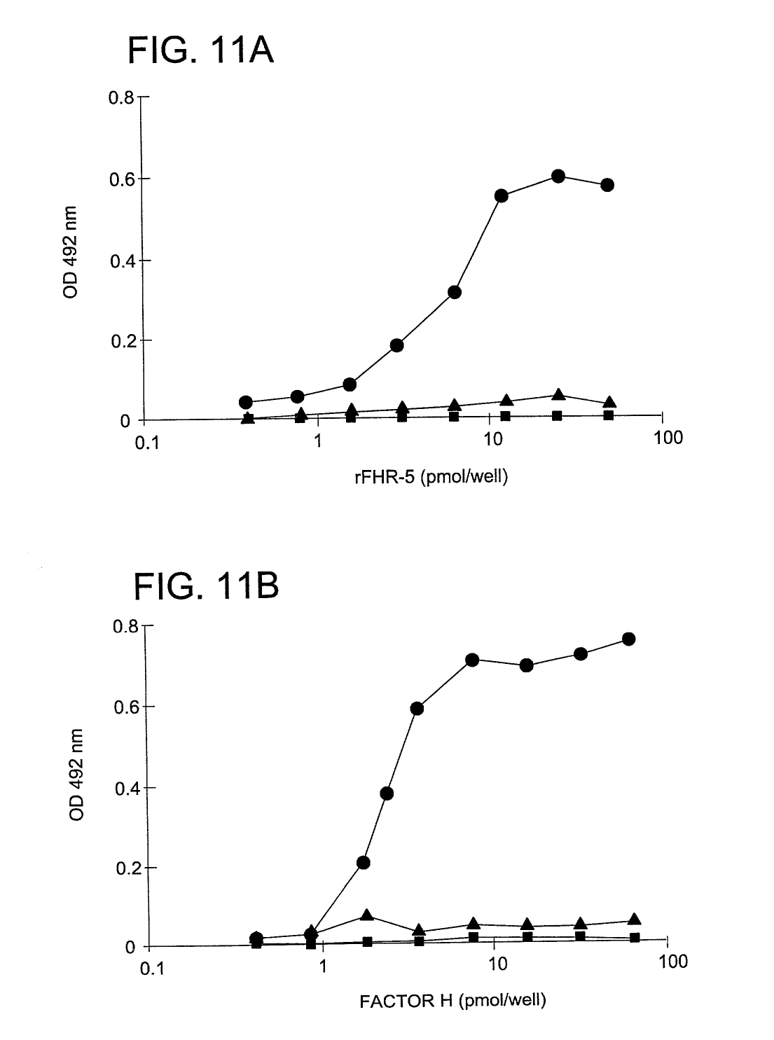

The results are shown in FIG. 11. Like Factor H (panel B, FIG. 11), C3b bound to FHR-5 (panel A, FIG. 11) in a dose-dependant and saturable manner. Neither human C5b-6 complexes nor HSA demonstrated any binding to FHR-5 or to Factor H.

### CONCLUSION

In light of the detailed description of the invention and the examples presented above, it can be appreciated that the several aspects of the invention are achieved.

It is to be understood that the present invention has been described in detail by way of illustration and example in order to acquaint others skilled in the art with the invention, its principles, and its practical application. Particular formulations and processes of the present invention are not limited to the descriptions of the specific embodiments presented, but rather the descriptions and examples should be viewed in terms of the claims that follow and their equivalents. While some of the examples and descriptions above include some conclusions about the way the invention may function, the inventor does not intend to be bound by those conclusions and functions, but puts them forth only as possible explanations.

It is to be further understood that the specific embodiments of the present invention as set forth are not intended as being exhaustive or limiting of the invention, and that many alternatives, modifications, and variations will be apparent to those of ordinary skill in the art in light of the foregoing examples and detailed description. Accordingly, this invention is intended to embrace all such alternatives, modifications, and variations that fall within the spirit and scope of the following claims.

What is claimed is:

1. A substantially purified factor H-related protein 5, comprising a primary amino acid sequence selected from the group consisting of:
a) SEQ ID NO: 2;
b) an amino acid sequence having at least 90% sequence identity to SEQ ID NO: 2;

2. A substantially purified factor H-related protein 5 fragment, comprising a fragment of SEQ ID NO: 2, wherein said fragment binds to complement component C3b and that when associated with activated complement is recognized by monoclonal antibody K2.254.

3. A substantially isolated polynucleotide comprising a nucleic acid sequence selected from the group consisting of:
a) a nucleic acid sequence that encodes SEQ ID NO:2;
b) a nucleic acid sequence that encodes an amino acid sequence having at least 90% sequence identity to SEQ ID NO: 2;
c) a nucleic acid sequence that encodes a fragment of SEQ ID NO: 2, wherein said fragment binds to complement component C3b and that when associated with activated complement is recognized by monoclonal antibody K2.254.;
d) SEQ ID NO: 1 or the complement thereof;
e) SEQ ID NO: 3 or the complement thereof;
f) a nucleic acid sequence that hybridizes to the nucleic acid sequence of a) under wash conditions of 0.1×SSPE, 0.1% SDS at 42° C. and encodes a protein that binds to complement component C3b and when associated with activated complement is recognized by monoclonal antibody K2.254;
g) a nucleic acid sequence of at least 20 nucleotides that hybridizes to the nucleic acid sequence of a) under wash conditions of 0.1×SSPE, 0.1% SDS at 42° C.;
h) a nucleic acid sequence that hybridizes to the nucleic acid sequence of b) under wash conditions of 0.1×SSPE, 0.1% SDS at 42° C. and encodes a protein that binds to complement component C3b and when associated with activated complement is recognized by monoclonal antibody K2.254; and
i) a nucleic acid sequence of at least 20 nucleotides that hybridizes to the nucleic acid sequence of b) under wash conditions of 0.1×SSPE, 0.1% SDS at 42° C.

4. A recombinant vector comprising the polynucleotide of claim 3.

5. The recombinant vector of claim 4, wherein said vector is a cloning vector.

6. The recombinant vector of claim 4, wherein said vector is an expression vector.

7. An expression cassette comprising a promoter, the polynucleotide of claim 3, and a transcription termination sequence.

8. A host cell comprising the vector of claim 4.

9. The host cell of claim 8, wherein said host cell is selected from the group consisting of bacterial cells, bacteriophage, yeast cells, insect cells, plant cells, and animal cells.

10. A host cell comprising the expression cassette of claim 7.

11. The host cell of claim 10, wherein said host cell is selected from the group consisting of bacterial cells, yeast cells, insect cells, plant cells, and animal cells.

12. An antibody that binds specifically binds to the factor H-related protein 5 of claim 1 and which is not monoclonal antibody K2.254.

13. The antibody of claim 12, wherein said antibody is a polyclonal antibody.

14. The antibody of claim 12, wherein said antibody is a monoclonal antibody.

15. The antibody of claim 12, wherein said antibody is a recombinant antibody.

16. The antibody of claim 15, wherein said antibody is a chimeric antibody.

17. The antibody of claim 16, wherein said antibody is a humanized antibody.

18. The antibody of claim 12, wherein said antibody is an immunologically active antibody fragment.

19. The antibody of claim 12, wherein said antibody binds to factor H-related protein 5 when factor H-related protein 5 is associated with activated complement.

20. An antibody that specifically binds to the factor H-related protein 5 fragment of claim 2 and which is not monoclonal antibody K2.254.

21. The antibody of claim 20, wherein said antibody is a polyclonal antibody.

22. The antibody of claim 20, wherein said antibody is a monoclonal antibody.

23. The antibody of claim 20, wherein said antibody is a recombinant antibody.

24. The antibody of claim 23, wherein said antibody is a chimeric antibody.

25. The antibody of claim 24, wherein said antibody is a humanized antibody.

26. The antibody of claim 20, wherein said antibody is an immunologically active antibody fragment.

27. The antibody of claim 12, wherein said antibody binds to factor said H-related protein 5 fragment when said factor H-related protein 5 fragment is associated with activated complement.

28. The antibody of claim 20, wherein said antibody binds to factor said H-related protein 5 fragment when said factor H-related protein 5 fragment is associated with activated complement.

29. A method for the detection of C5b-9complement complexes comprising contacting a cell or tissue with the antibody of claim 12 and detecting the binding of the antibody to the cell or tissue.

30. A method for the detection of C5b-9complement complexes comprising contacting a cell or tissue with the antibody of claim 20 and detecting the binding of the antibody to the cell or tissue.


SEQUENCE LISTING (40 sequences)

SEQ ID NO: 1 - DNA, 2823 bp, Homo sapiens
ggcaggtgcttgttactgttaatgaaagcagatttaaagcaacaccaccatcactggagtatttttagttatatacgattgagactaccaagcatgttgctcttattcagtgtaatcctaatctcatgggtatccactgttgggggagaaggaacactttgtgattttccaaaaatacaccatggatttctgtatgatgaagaagattataaccctttttcccaagttcctacaggggaagttttctattactcctgtgaatataattttgtgtctccttcaaaatccttttggactcgcataacatgcacagaagaaggatggtcaccaacaccgaagtgtctcagaatgtgttcctttccttttgtgaaaaatggtcattctgaatcttcaggactaatacatctggaaggtgatactgtacaaattatttgcaacacaggatacagccttcaaaacaatgagaaaaacatttcgtgtgtagaacggggctggtccactcctcccatatgcagcttcactaaaggagaatgtcatgttccaattttagaagccaatgtagatgctcagccaaaaaaagaaagctacaaagttggagacgtgttgaaattctcctgcagaaaaaatcttataagagttggatcagactcagttcaatgttaccaatttgggtggtcacctaactttccaacatgcaaaggacaagtacgatcatgtggtccacctcctcaactctccaatggtgaagttaaggagataagaaaagaggaatatggacacaatgaagtagtggaatatgattgcaatcctaattttataataaacgggcctaagaaaatacaatgtgtggatggagaatggacaactttacccacttgtgttgaacaagtgaaaacatgtggatacatacctgaactcgagtacggttatgttcagccgtctgtccctccctatcaacatggagtttcagtcgaggtgaattgcagaaatgaatatgcaatgattggaaataacatgattacctgtattaatggaatatggacagagcttcctatgtgtgttgcaacacaccaacttaagaggtgcaaaatagcaggagttaatataaaaacattactcaagctatctgggaaagaatttaatcataattctagaatacgttacagatgttcagacatcttcagatacaggcactcagtctgtataaacgggaaatggaatcctgaagtagactgcacagaaaaaagggaacaattctgcccaccgccacctcagatacctaatgctcagaatatgacaaccacagtgaattatcaggatggagaaaaagtagctgttctctgtaaagaaaactatctacttccagaagcaaaagaaattgtatgtaaagatggacgatggcaatcattaccacgctgtgttgagtctactgcatattgtgggccccctccatctattaacaatggagataccacctcattcccattatcagtatatcctccagggtcaacagtgacgtaccgttgccagtccttctataaactccagggctctgtaactgtaacatgcagaaataaacagtggtcagaaccaccaagatgcctagatccatgtgtggtatctgaagaaaacatgaacaaaaataacatacagttaaaatggagaaacgatggaaaactctatgcaaaaacaggggatgctgttgaattccagtgtaaattcccacataaagcgatgatatcatcaccaccatttcgagcaatctgtcaggaagggaaatttgaatatcctatatgtgaatgaagcaagcataattttcctgaatatattcttcaaacatccatctacgctaaaagtagccattatgtagccaattctgtagttacttcttttattctttcaggtgttgtttaactcagttttatttagaactctggatttttagagctttagaaatttgtaagctgagagaacaatgtttcacttaataggagggtgtcttagtccatattacattgttataacagagtatcacagactggataacttctaaccaatagtttatttgtttcataaatctaaaagctgagaagtccaagatggtggggctgcctctggtgagggtcttctcgaagcatcataatatgctggaaggcatcacaacatggtggaagggatcacgtggcaaaagagcatgtacatgggagtgagagaaaaagagagagagagacagagtggcgggggccggggaggagcgcaaactcatcctttataaagacaccactcctgagataacaatccaatcccatgataatgacattaatccattcaagaagatagagctctcgtgacttaatcaccttctaaagatctcacctgacaacactgttgcattggcagttaagtttccacgtaaactttcggggacacattcaaaccacaggagaaactcaaattgttcctgggcaaatcacaacatggggaattttattcataaatgtccacagaaacagtaaatgttctcgcttcagaacttaattcatctaatccctcctgtttgtctcaaattataggataactttgaaactttctgaattaacgttatttaaaaggaaatgtagatgttattttagtctctatcttcaggttattatcacttaaaaacctgcgaaagctgtcaacttttgtggttgtagcaagtattaataaatatttataaatcctctaatgtaagtctagctacctatccaatactaaataccccttaaagtattaaatgcactatctgctgtaaacggaaaaaaaaaaaaaaaaaaaaaaaaa

SEQ ID NO: 2 - protein, 569 aa, Homo sapiens
MLLLFSVILISWVSTVGGEGTLCDFPKIHHGFLYDEEDYNPFSQVPTGEVFYYSCEYNFVSPSKSFWTRITCTEEGWSPTPKCLRMCSFPFVKNGHSESSGLIHLEGDTVQIICNTGYSLQNNEKNISCVERGWSTPPICSFTKGECHVPILEANVDAQPKKESYKVGDVLKFSCRKNLIRVGSDSVQCYQFGWSPNFPTCKGQVRSCGPPPQLSNGEVKEIRKEEYGHNEVVEYDCNPNFIINGPKKIQCVDGEWTTLPTCVEQVKTCGYIPELEYGYVQPSVPPYQHGVSVEVNCRNEYAMIGNNMITCINGIWTELPMCVATHQLKRCKIAGVNIKTLLKLSGKEFNHNSRIRYRCSDIFRYRHSVCINGKWNPEVDCTEKREQFCPPPPQIPNAQNMTTTVNYQDGEKVAVLCKENYLLPEAKEIVCKDGRWQSLPRCVESTAYCGPPPSINNGDTTSFPLSVYPPGSTVTYRCQSFYKLQGSVTVTCRNKQWSEPPRCLDPCVVSEENMNKNNIQLKWRNDGKLYAKTGDAVEFQCKFPHKAMISSPPFRAICQEGKFEYPICE

SEQ ID NO: 3 - DNA, 1707 bp, Artificial Sequence
  Generic sequence
atgytnytnytnttywsngtnathytnathwsntgggtnwsnacngtnggnggngarggnacnytntgygayttyccnaarathcaycayggnttyytntaygaygargargaytayaayccnttywsncargtnccnacnggngargtnttytaytaywsntgygartayaayttygtnwsnccnwsnaarwsnttytggacnmgnathacntgyacngargarggntggwsnccnacnccnaartgyytnmgnatgtgywsnttyccnttygtnaaraayggncaywsngarwsnwsnggnytnathcayytngarggngayacngtncarathathtgyaayacnggntaywsnytncaraayaaygaraaraayathwsntgygtngarmgnggntggwsnacnccnccnathtgywsnttyacnaarggngartgycaygtnccnathytngargcnaaygtngaygcncarccnaaraargarwsntayaargtnggngaygtnytnaarttywsntgymgnaaraayytnathmgngtnggnwsngaywsngtncartgytaycarttyggntggwsnccnaayttyccnacntgyaarggncargtnmgnwsntgyggnccnccnccncarytnwsnaayggngargtnaargarathmgnaargargartayggncayaaygargtngtngartaygaytgyaayccnaayttyathathaayggnccnaaraarathcartgygtngayggngartggacnacnytnccnacntgygtngarcargtnaaracntgyggntayathccngarytngartayggntaygtncarccnwsngtnccnccntaycarcayggngtnwsngtngargtnaaytgymgnaaygartaygcnatgathggnaayaayatgathacntgyathaayggnathtggacngarytnccnatgtgygtngcnacncaycarytnaarmgntgyaarathgcnggngtnaayathaaracnytnytnaarytnwsnggnaargarttyaaycayaaywsnmgnathmgntaymgntgywsngayathttymgntaymgncaywsngtntgyathaayggnaartggaayccngargtngaytgyacngaraarmgngarcarttytgyccnccnccnccncarathccnaaygcncaraayatgacnacnacngtnaaytaycargayggngaraargtngcngtnytntgyaargaraaytayytnytnccngargcnaargarathgtntgyaargayggnmgntggcarwsnytnccnmgntgygtngarwsnacngcntaytgyggnccnccnccnwsnathaayaayggngayacnacnwsnttyccnytnwsngtntayccnccnggnwsnacngtnacntaymgntgycarwsnttytayaarytncarggnwsngtnacngtnacntgymgnaayaarcartggwsngarccnccnmgntgyytngayccntgygtngtnwsngargaraayatgaayaaraayaayathcarytnaartggmgnaaygayggnaarytntaygcnaaracnggngaygcngtngarttycartgyaarttyccncayaargcnatgathwsnwsnccnccnttymgngcnathtgycargarggnaarttygartayccnathtgygar

SEQ ID NO: 4 - DNA, 29 bp, Artificial Sequence
  GSP-1 Primer
ggtgtgttgcaacacacataggaagctct

SEQ ID NO: 5 - DNA, 28 bp, Artificial Sequence
  GSP-2 Primer
gtcatgttgcccattttagaagccaatg

SEQ ID NO: 6 - DNA, 20 bp, Artificial Sequence
  CAP-F1 Primer
ggagaaggaacactttgtga

SEQ ID NO: 7 - DNA, 20 bp, Artificial Sequence
  CAP-F2 Primer
ataagagttggatcagactc

SEQ ID NO: 8 - DNA, 20 bp, Artificial Sequence
  CAP-F3 Primer
gtatatcctccagggtcaac

SEQ ID NO: 9 - DNA, 21 bp, Artificial Sequence
  CAP-F4 Primer
gtggatacatacctgaactcg

SEQ ID NO: 10 - DNA, 21 bp, Artificial Sequence
  CAP-F5 Primer
tcaccaacaccgaagtgtctc

SEQ ID NO: 11 - DNA, 21 bp, Artificial Sequence
  CAP-F6 Primer
ccatctacgctaaaagtagcc

SEQ ID NO: 12 - DNA, 19 bp, Artificial Sequence
  CAP-R1 Primer
gaacataaccgtactcgag

SEQ ID NO: 13 - DNA, 19 bp, Artificial Sequence
  CAP-R2 Primer
ccacatgatcgtacttgtc

SEQ ID NO: 14 - DNA, 19 bp, Artificial Sequence
  CAP-R3 Primer
gagcatctacattggcttc

SEQ ID NO: 15 - DNA, 20 bp, Artificial Sequence
  CAP-R4 Primer
gttgaccctggaggatatac

SEQ ID NO: 16 - DNA, 21 bp, Artificial Sequence
  CAP-R5 Primer
ctgagcattaggtatctgagg

SEQ ID NO: 17 - DNA, 23 bp, Artificial Sequence
  CAP-R6 Primer
cttcccctgtacgaacttgggaa

SEQ ID NO: 18 - DNA, 24 bp, Artificial Sequence
  707-F2 Primer
gttcagacatcttcagatacaggc

SEQ ID NO: 19 - DNA, 23 bp, Artificial Sequence
  707-F3 Primer
cagttaaaatggagaaacgatgg

SEQ ID NO: 20 - DNA, 29 bp, Artificial Sequence
  3′BAC Primer
ggactagtggctacttttagcgtagatgg

SEQ ID NO: 21 - DNA, 23 bp, Artificial Sequence
  5′BAC(C) Primer
gaaggaacactttgtgattttcc

SEQ ID NO: 22 - DNA, 26 bp, Artificial Sequence
  TCAP-F Primer
cgggcaggtgcttgttactgttaatg

SEQ ID NO: 23 - DNA, 25 bp, Artificial Sequence
  TCAP-R Primer
gctacaaccacaaaagttgacagct

SEQ ID NO: 24 - DNA, 25 bp, Artificial Sequence
  Mlu 1-F Primer
cgacgcgtgaaggaacactttgtga

SEQ ID NO: 25 - DNA, 29 bp, Artificial Sequence
  Sal 1-R Primer
gccgtcgacccttcacatataggatattc

SEQ ID NO: 26 - DNA, 20 bp, Artificial Sequence
  254 F-1 Primer
ggngartgycaygtnccnat

SEQ ID NO: 27 - DNA, 20 bp, Artificial Sequence
  254 R-1 Primer
cartcnacytcnggrttcca

SEQ ID NO: 28 - protein, 10 aa, Artificial Sequence
  Homology with human Factor H protein amino acids 416-423
WNPEVDCTEK

SEQ ID NO: 29 - protein, 17 aa, Artificial Sequence
  Homology with human Factor H protein amino acids 549-565
GECHVPILEANVDAQPK

SEQ ID NO: 30 - protein, 7 aa, Artificial Sequence
  Homology with human Factor H protein amino acids 1205-1211
FEYPICE

SEQ ID NO: 31 - protein, 23 aa, Artificial Sequence
  Homology with human Factor H protein amino acids 629-651
EEYGHNEVVEYDCNPNFIINGPK

SEQ ID NO: 32 - protein, 15 aa, Artificial Sequence
  Homology with human Factor H protein amino acids 653-672
IQCVDGEWTTLPTCV

SEQ ID NO: 33 - protein, 5 aa, Artificial Sequence
  Homology with human FHR 1/2 protein amino acids 115-119
GWSTP

SEQ ID NO: 34 - protein, 12 aa, Artificial Sequence
  Homology with human FHR-2 protein amino acids 47-58
SFWTRITCTEEG

SEQ ID NO: 35 - protein, 5 aa, Artificial Sequence
  Homology with human SCR 2 FHR-5 protein
MCSFP

SEQ ID NO: 36 - protein, 16 aa, Artificial Sequence
  Homology with human SCR20 FHR-5 protein
AMISSPPFRAICQEGK

SEQ ID NO: 37 - protein, 10 aa, Artificial Sequence
  Homology with human Factor H protein amino acids 1175-1184
TGDAVEFQCK

SEQ ID NO: 38 - protein, 9 aa, Artificial Sequence
  Homology with human Factor H protein amino acids 837-845
DGRWQSLPR

SEQ ID NO: 39 - protein, 6 aa, Artificial Sequence
  Homology with human Factor H protein amino acids 817-822
VALVCK

SEQ ID NO: 40 - protein, 9 aa, Artificial Sequence
  Homology with human Factor H protein amino acids 823-831
ENYLLPEAK